(19) United States
(12) Patent Application Publication
(10) Pub. No.: US 2023/0404981 A1
(43) Pub. Date: Dec. 21, 2023

(54) PHARMACEUTICALLY ACTIVE COMPOUND FORMULATIONS

(71) Applicant: The University of Liverpool, Liverpool, Merseyside (GB)

(72) Inventors: Steven Paul Rannard, Mickle Trafford, Chesire (GB); Andrew Owen, Liverpool, Merseyside (GB); James Hobson, Liverpool, Merseyside (GB); Alison Savage, Liverpool, Merseyside (GB); Andrew Dwyer, Hoylake (GB); Catherine Unsworth, Liverpool, Merseyside (GB); Thomas Oliver McDonald, Liverpool, Merseyside (GB)

(21) Appl. No.: 18/252,508

(22) PCT Filed: Nov. 10, 2021
(86) PCT No.: PCT/GB2021/052909

§ 371 Date: May 10, 2023

(30) Foreign Application Priority Data
Nov. 10, 2020 (GB) 2017724.2

(51) Int. Cl. A61K 31/426; A61K 9/16; A61K 31/167

(52) U.S. Cl. CPC A61K 31/426; A61K 9/1623; A61K 9/1641; A61K 9/1635; A61K 31/167

(57) ABSTRACT

The present invention relates to solid compositions of pharmaceutically active compounds, aqueous dispersions derived from these compositions and processes for the preparation of these solid compositions and dispersions. The present invention also relates to pharmaceutical compositions derived from these solid compositions and dispersions, and their use in the treatment and/or prophylaxis of helminthic, protozoal, and viral infections.

### FIELD OF THE INVENTION

The present invention relates to solid compositions of pharmaceutically active compounds, aqueous dispersions derived from these compositions and processes for the preparation of these solid compositions and dispersions. The present invention also relates to pharmaceutical compositions derived from these solid compositions and dispersions, and their use in the treatment and/or prophylaxis of helminthic, protozoal, and viral infections.

### BACKGROUND

Since early 2020, COVID 19, a viral disease caused by infection with the SARS-CoV-2 virus, has spread throughout the world, with a pandemic being declared by the World Health Organisation on 11 Mar. 2020. SARS-CoV-2 initially infects the upper respiratory tract, provoking few symptoms, and may then spread to the lower respiratory tract, causing more serious symptoms such as pneumonia and, in the worst cases, death.

As SARS-CoV-2 is a novel coronavirus, there are no specific treatments available, nor prophylaxis. Significant research efforts have begun to develop treatments (including monoclonal antibodies and novel small-molecules) and vaccines to treat and prevent COVID 19. However, these approaches, even when being ‘fast-tracked’ from a regulatory perspective, may take years to come to fruition, and, as completely new technologies, will have issues being scaled up to address the world-wide nature of the pandemic, leaving a significant medical and societal need in the short term.

In response to this need, there has been a great deal of highly publicised research looking to repurpose existing drugs. These approaches have the advantage of using drugs that have cleared a number of hurdles as they have known safety and pharmacokinetic compounds, and, in many cases, have well-known and scalable production processes. The difficulty with these approaches lies in selecting known drugs that may be active against SARS-CoV-2.

Two of the most publicised repurposing approaches have utilised hydroxychloroquine (an antimalarial) and remdesivir (a proprietary antiviral). The effectiveness of the former has not been conclusively demonstrated, while the latter has production bottlenecks, leading to high cost for a course of treatment, and requires IV administration, making them unsuitable for community therapy and prophylaxis.

For a given drug candidate to be effective against a virus, such as SARS-CoV-2, it is required to achieve a minimum concentration in the human body that is equal to, or greater than, the concentration required to inhibit the virus. Therefore, one rationale for the selection of candidates for repurposing efforts is to compare the ratio between the concentration of the drug required to inhibit SARS-CoV-2 in vitro (often measured as EC₉₀), with the concentration of the drug that can be obtained in vivo (often measured as Cₘₐₓ). Both of these parameters are known for a number of drugs and the above comparison has been made in Clin. Phamacol. Ther., 2020, DOI: 10.1002/cpt.1909, the findings of which are summarised in FIG. 1. Simply put, for a candidate to have a high likelihood of success, it is required to achieve concentrations capable of inhibiting SARS-CoV-2 in the body. In other words, the Cₘₐₓ/EC₉₀ ratio needs to be greater than 1. It should be noted that it is the free drug Cₘᵢₙ that is needed to be greater than the EC₉₀ value to provide effective inhibition of SARS-CoV-2, but Cₘₐₓ was used in the analysis as a proxy as it is more commonly recorded. In any case, it would be advantageous to increase the ratio of Cₘₐₓ (or Cₘᵢₙ) to EC₉₀ through formulation. Of the candidates meeting this criteria, niclosamide and nitazoxanide are of particular interest as they are well tolerated, widely available and inexpensive, making them suitable for the treatment of large populations.

Niclosamide is a well known anthelmintic, commonly used for the treatment for tapeworm, and is taken orally. Niclosamide has also been investigated for anticancer and bronchiodilator applications.

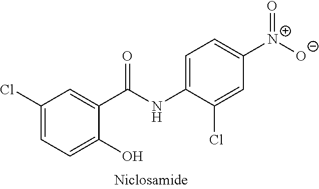 [SMILES: O=C(Nc1ccc([N+](=O)[O-])cc1Cl)c1cc(Cl)ccc1O]

Niclosamide is an example of the salicylanilide class of drugs, which are well-known for their use as anthelmintic drugs and as antiseptics. There has also been much interest in their potential for treating viral infections, fungal infections, and cancers in recent years. Salicylanilide drugs are based on salicylanilide, with various substitutions, primarily halogens, on the aryl groups. Without wishing to be bound by theory, it seems likely, given the similarities in structure and mechanisms of action between niclosamide and other members of the salicylanilide class of drugs, that other members of the salicylanilide class of drugs will also be useful in the present invention.

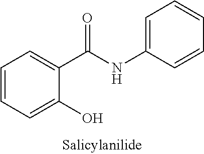 [SMILES: O=C(Nc1ccccc1)c1ccccc1O]

Nitazoxanide is a broad spectrum antiparasitic and antiviral of the thiazolide class, and is taken orally. The active metabolite of nitazoxanide is tizoxanide, produced by hydrolysis of the acetyl group, and is an antiparasitic drug in its own right. Nitazoxanide has also been investigated for anticancer and bronchiodilator applications.

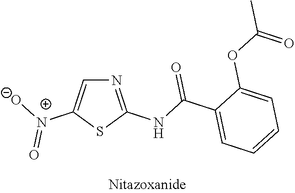 [SMILES: CC(=O)Oc1ccccc1C(=O)Nc1ncc([N+](=O)[O-])s1]

Nitazoxanide is an example of the thiazolide class of drugs, which are well known for their use as anthelmintic drugs. In particular nitazoxanide and alternative prodrugs of tizoxanide are under investigation for the treatment of many viral diseases and cancers. Exemplary tizoxanide prodrugs would be molecules with the above structure in which the acetyl ester is replaced with an alternative ester. Alternative esters could be drawn from alkyl esters (such as propionyl ester, butyryl ester, isobutyryl ester, pentanoyl ester, isopentanoyl ester, neopentanoyl ester), aryl esters (such as benzoyl ester, or substituted benzoyl esters), or esters of amino acids. For further examples, see WO2016/077420A1, incorporated herein by reference. Without wishing to be bound by theory, it seems likely, given the similarities in structure and mechanisms of action between nitazoxanide and other members of the thiazolide class of drugs, that other members of the salicylanilide class of drugs will also be useful in the present invention. This seems to be especially true for other tizoxanide prodrugs.

However, despite being amongst the most pharmaceutically active compounds against SAR-CoV-2 and achieving an acceptable Cₘₐₓ/EC₉₀ ratio, when orally dosed nitazoxanide and niclosamide are not certain to achieve effective distribution to the tissues most affected by SARS-CoV-2: the upper and lower respiratory tract. Improving distribution to these tissues would also be beneficial when seeking to use these compounds, and others in their classes, against other diseases with respiratory involvement, for example, other viral infections (such as influenza, SARS, and MERS), helminth infections (such as lungworm), and protozoal infections. For example, the methodology that demonstrated the applicability of these compounds to SARS-CoV-2 has also been used to determine a suitable oral dosing regimen for influenza (medRxiv preprint doi: https://doi.org/10.1101/2020.05.01.20087130).

Many of the identified pharmaceutically active compounds are poorly soluble in water (as low as 7.99 and 7.55 μg/mL for niclosamide and nitazoxanide respectively) and a wide range of other solvents, making them difficult to formulate and limiting the methods by which they may be administered to patients. These difficulties also extend to many other members of the salicylanilide and thiazolide classes of drugs.

It is the object of the present invention to solve at least one of the above indicated problems. In particular, it is the object of the present invention to provide compositions enabling the delivery of candidates to the target tissues (e.g. upper and lower respiratory tract) in high enough concentrations to effect treatment and/or prophylaxis of viral infections, for example coronavirus infections such as COVID-19, helminthic infections, and/or protozoal infections.

### SUMMARY OF THE INVENTION

A first aspect of the present invention provides a solid composition comprising a plurality of nanoparticles of a pharmaceutically active compound dispersed within a carrier material comprising at least one hydrophilic polymer and at least one sugar, wherein the pharmaceutically active compound is selected from nitazoxanide and niclosamide.

The at least one hydrophilic polymer may be selected from polyvinyl alcohols, polyvinylpyrrolidones, poloxamers, hydroxypropyl celluloses, and hydroxypropyl methyl celluloses. Sometimes, the at least one hydrophilic polymer is selected from polyvinyl alcohols, poloxamers, hydroxypropyl celluloses, and hydroxypropyl methyl celluloses.

The at least one sugar may be selected from monosaccharides, disaccharides, and oligosaccharides, preferably the at least one sugar is a disaccharide such as sucrose or lactose.

In one embodiment of the solid composition, the pharmaceutically active compound is nitazoxanide; the at least one hydrophilic polymer is poloxamer; and the at least one sugar is selected from sucrose or lactose.

Preferably the solid composition comprises 50 to 60 wt % nitazoxanide; 10 to 30 wt % poloxamer; and 10 to 30 wt % sucrose or lactose. More preferably, the solid composition comprises 50 wt % nitazoxanide; 20 to 30 wt % poloxamer; and 20 to 30 wt % lactose.

Preferably the solid composition comprises 60 wt % nitazoxanide; 10 to 30 wt % poloxamer; and 10 to 30 wt % sucrose.

In one embodiment of the solid composition the pharmaceutically active compound is niclosamide; the at least one hydrophilic polymer is hydroxypropyl methyl cellulose; and the at least one sugar is sucrose. Preferably, the solid composition comprises 50 to 70 wt % niclosamide; 15 to 25 wt % hydroxypropyl methyl cellulose; and 15 to 25 wt % sucrose.

In one embodiment of the solid composition the pharmaceutically active compound is niclosamide; the at least one hydrophilic polymer is polyvinylpyrrolidone; and the at least one sugar is sucrose or lactose. Preferably, the solid composition comprises 40 to 70 wt % niclosamide; 10 to 30 wt % polyvinylpyrrolidone; and 15 to 40 wt % sucrose or lactose. More, preferably, the solid composition comprises 50 to 70 wt % niclosamide; 15 to 25 wt % polyvinylpyrrolidone; and 15 to 25 wt % sucrose or lactose.

A second aspect of the present invention provides a process for preparing a solid composition according to the first aspect of the present invention, the method comprising the steps of: (a) providing an active solution comprising the pharmaceutically active compound in a water-miscible solvent; (b) providing a carrier material solution comprising one or more hydrophilic polymers and one or more sugars in an aqueous solvent; (c) mixing the solutions prepared in steps (a) and (b); and (d) removing the mixed solvent to produce the solid composition; wherein the pharmaceutically active compound is selected from nitazoxanide or niclosamide.

The at least one hydrophilic polymer may be selected from polyvinyl alcohol, polyvinylpyrrolidone, poloxamers, hydroxypropyl cellulose, and hydroxypropyl methyl cellulose. Sometimes, the at least one hydrophilic polymer is selected from polyvinyl alcohols, poloxamers, hydroxypropyl celluloses, and hydroxypropyl methyl celluloses.

The at least one sugar may be selected from monosaccharides, disaccharides, and oligosaccharides, preferably the at least one sugar is a disaccharide, such as sucrose or lactose.

The water-miscible solvent may be selected from dimethylsulfoxide, acetone, butanone, ethanol, or mixtures thereof. Sometimes, the water-miscible solvent is selected from dimethylsulfoxide, acetone, ethanol, or mixtures thereof.

The active solution may be maintained at an elevated temperature prior to the mixing step.

The step of mixing the active solution and the carrier material solution may additionally comprise homogenising and/or sonicating the dispersion.

The active solution and the carrier material solution may be mixed in a ratio of about 1:9 to about 1:2.

The step of removing the mixed solvent may comprise spray-drying.

In one embodiment, the pharmaceutically active compound is nitazoxanide; the at least one hydrophilic polymer is poloxamer; the at least one sugar is sucrose and/or lactose; and the water miscible solvent is dimethylsulfoxide.

In one embodiment, the pharmaceutically active compound is niclosamide; the at least one hydrophilic polymer is hydroxypropyl methyl cellulose or polyvinylpyrrolidone; the at least one sugar is sucrose or lactose; and the water miscible solvent is a mixture of ethanol and acetone or is a mixture of ethanol and butanone.

In another embodiment, the pharmaceutically active compound is niclosamide; the at least one hydrophilic polymer is hydroxypropyl methyl cellulose; the at least one sugar is sucrose; and the water miscible solvent is a mixture of ethanol and acetone.

A third aspect of the present invention provides a pharmaceutical composition comprising a solid composition according to the first aspect of the invention, and optionally one or more pharmaceutically acceptable excipients.

The pharmaceutical composition may be a dry inhalable powder suitable for use with a dry powder inhaler.

The pharmaceutical may be a suspension of the solid composition, and optionally one or more pharmaceutically acceptable excipients, in a volatile propellant suitable for use with a pressurised metered-dose inhaler.

A fourth aspect of the present invention provides a solid composition according to the first aspect of the present invention, or a pharmaceutical composition according the third aspect of the present invention, for use as a medicament.

A fifth aspect of the present invention provides a solid composition according to the first aspect of the present invention, or a pharmaceutical composition according the third aspect of the present invention, for use in the treatment and/or prevention of viral infection, helminth infection, or protozoal infection, optionally wherein the viral infection is a coronavirus infection, such as SARS-CoV-2 infection.

A sixth aspect of the present invention provides a method of treating and/or preventing a viral infection, helminth infection, or protozoal infection, optionally wherein the viral infection is a coronavirus infection, such as SARS-CoV-2 infection, the method comprising administering a therapeutically effective amount of a solid composition according to the first aspect of the present invention, or a pharmaceutical composition according the third aspect of the present invention, to a patient suffering from, or at risk of suffering from, the viral infection.

A seventh aspect of the present invention provides aqueous dispersion comprising a plurality of nanoparticles of one or more pharmaceutically active compounds dispersed within an aqueous medium, wherein the pharmaceutically active compound is selected from nitazoxanide and niclosamide, each nanoparticle being stabilised by the one or more hydrophilic polymers and/or the one or more sugars adsorbed to the surface of the nanoparticle.

The aqueous phase may comprise water, saline, or phosphate buffered saline. According to some embodiments, the aqueous phase comprises saline.

The concentration of the pharmaceutically active compound in the dispersion may be in the range of 1 to 800 mg/mL, preferably 10 to 600 mg/mL, more preferably 225 to 575 mg/mL, most preferably 300 to 500 mg/mL Alternatively, the total solids content of the dispersion may be in the range of 1 to 10 mg/mL.

An eighth aspect of the present invention provides a process for the preparation of an aqueous dispersion according to the seventh aspect of the present invention, the process comprising dispersing a solid composition according to the first aspect of the present invention in an aqueous medium.

A ninth aspect of the present invention provides a pharmaceutical composition comprising an aqueous dispersion according to the seventh aspect of the present invention, and optionally one or more pharmaceutically acceptable excipients.

A tenth aspect of the present invention provides an aqueous dispersion according to the eighth aspect of the present invention, or a pharmaceutical composition according to the ninth aspect of the present invention, for use as a medicament.

An eleventh aspect of the present invention provides an aqueous dispersion according to the eighth aspect of the present invention, or a pharmaceutical composition according to the ninth aspect of the present invention, for use in the treatment and/or prevention of viral infection, helminth infection, or protozoal infection, optionally wherein the viral infection is a coronavirus infection, such as SARS-CoV-2 infection.

A twelfth aspect of the present invention provides a method of treating and/or preventing a viral infection, helminth infection, or protozoal infection, optionally wherein the viral infection is a coronavirus infection, such as SARS-CoV-2 infection, the method comprising administering a therapeutically effective amount of an aqueous dispersion according to the eighth aspect of the present invention, or a pharmaceutical composition according to the ninth aspect of the present invention, to a patient suffering from, or at risk of suffering from, the viral infection.

A thirteenth aspect of the present invention provides an intramuscularly-injectable pharmaceutically active compound formulation or a subcutaneously-injectable pharmaceutically active compound formulation comprising a solid composition according to the first aspect of the present invention, an aqueous dispersion according to the eighth aspect of the present invention, or a pharmaceutical composition according to the ninth aspect of the present invention.

The intramuscularly-injectable pharmaceutically active compound formulation of the thirteenth aspect of the present invention, or the subcutaneously-injectable pharmaceutically active compound formulation according to the thirteenth aspect of the present invention, in depot form.

A fourteenth aspect of the present invention provides an intramuscularly-injectable pharmaceutically active compound formulation according to the thirteenth aspect of the present invention, or a subcutaneously-injectable pharmaceutically active compound formulation according to the thirteenth aspect of the present invention 38, for use as a medicament.

A fifteenth aspect of the present invention provides an intramuscularly-injectable pharmaceutically active compound formulation according to the thirteenth aspect of the present invention, or a subcutaneously-injectable pharmaceutically active compound formulation according to the thirteenth aspect of the present invention, for use in the treatment and/or prevention of viral infection, helminth infection, or protozoal infection, optionally wherein the viral infection is a coronavirus infection, such as SARS-CoV-2 infection.

A sixteenth aspect of the present invention provides a method of treating and/or preventing a viral infection, helminth infection, or protozoal infection, optionally wherein the viral infection is a coronavirus infection, such as SARS-CoV-2 infection, the method comprising administering a therapeutically effective amount of an intramuscularly-injectable pharmaceutically active compound formulation according to the thirteenth aspect of the present invention, or a subcutaneously-injectable pharmaceutically active compound formulation according to the thirteenth aspect of the present invention, to a patient suffering from, or at risk of suffering from, the viral infection.

### BRIEF DESCRIPTION OF THE DRAWINGS

FIG. 1 is a bar graph showing the Cₘₐₓ/EC₉₀ (with respect to SARS-CoV-2) ratio for a range of drugs. The top 14 entries (tipranavir, nitazoxanide, niclosamide, nelfinavir, remdesivir, favipiravir, eltrombopag, lopinavir, ritonavir, mefloquine, chloroquine, and anidulafungin, with some repetitions) display a ratio of over 1. The top seven entries (tipranavir, nitazoxanide, niclosamide, nelfinavir, remdesivir, favipiravir, and eltrombopag) display a ratio of over 2

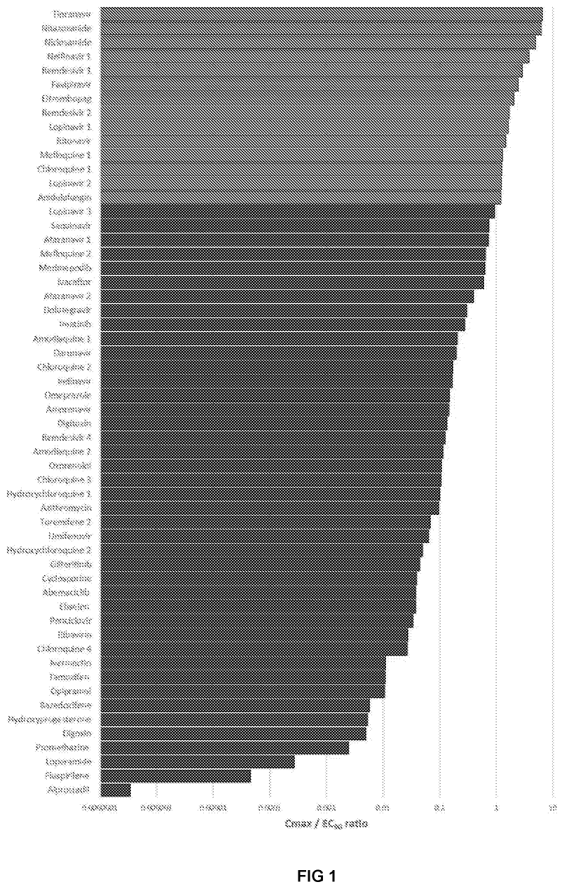

FIG. 2 is a series of plots of the plasma concentration (in ng/mg) of niclosamide in Sprague Dawley rats over a period of 14 days following intramuscular injections of (from left to right) 50, 100, and 200 mg/kg of niclosamide. The niclosamide that was administered was in the form of an aqueous dispersion produced by dispersing an appropriate quantity of the niclosamide/HPMC/sucrose (60/20/20 wt %) formulation in a vehicle of 20 wt % HPMC, 20 wt % sucrose and 60 wt % water to arrive at concentrations of 25, 50, and 100 mg/kg.

### DETAILED DESCRIPTION

### Definitions

In some embodiments, the term “pharmaceutically active compound” is used herein to refer to compounds found to have a Cₘₐₓ/EC₉₀ (with respect to SARS-CoV-2) ratio in excess of 1, such as tipranavir, nitazoxanide, niclosamide, nelfinavir, remdesivir, favipiravir, eltrombopag, lopinavir, ritonavir, mefloquine, chloroquine, and anidulafungin. In preferred embodiments, the term “pharmaceutically active compound” is used herein to refer to compounds found to have a Cₘₐₓ/EC₉₀ (with respect to SARS-CoV-2) ratio in excess of 2, such as tipranavir, nitazoxanide, niclosamide, nelfinavir, remdesivir, favipiravir, and eltrombopag.

The term “salicylanilide” is used herein to refer to drugs in the salicylanilide class (i.e. 2-Hydroxy-N-phenylbenzamide derivatives), and includes pharmaceutically acceptable prodrugs, salts and solvates thereof, as well as any polymorphic or amorphous forms thereof. Members of this class of drugs, such as niclosamide, have been found to have anthelminthic, antiprotozoal, and antiviral activity. Exemplary salicylanilides include niclosamide, oxyclozanide, rafoxanide, and bromochlorosalicylanilide.

The term “niclosamide” is used herein to refer to the compound with IUPAC name 5-chloro-N-(2-chloro-4-nitrophenyl)-2-hydroxybenzamide, and includes pharmaceutically acceptable salts and solvates thereof, as well as any polymorphic or amorphous forms thereof.

The term “thiazolide” is used herein to refer to drugs in the thiazolide class (i.e. 2-thiazolyl benzamide derivatives), and includes pharmaceutically acceptable prodrugs, salts and solvates thereof, as well as any polymorphic or amorphous forms thereof. Members of this class of drugs, such as tizoxanide prodrugs, have been round to have anthelminthic, antiprotozoal, and antiviral activity.

The term “tizoxanide prodrug” is used herein to refer to prodrugs of tizoxanide (i.e. the compound with IUPAC name 2-Hydroxy-N-(5-nitro-2-thiazolyl)benzamide), and includes pharmaceutically acceptable salts and solvates thereof, as well as any polymorphic or amorphous forms thereof. In many cases, the prodrug is formed through esterification of the hydroxyl to form, for example, an alkyl ester, aryl ester, or the ester of an amino acid.

The term “nitazoxanide” is used herein to refer to the compound with IUPAC name [2-[(5-Nitro-1,3-thiazol-2-yl)carbamoyl]phenyl]ethanoate, and includes pharmaceutically acceptable salts and solvates thereof, as well as any polymorphic or amorphous forms thereof.

The term “viral infection” is used herein to refer to viral infections in general, including by SARS-CoV-2 and other animal and human coronaviruses. Although the initial investigation is in the context of COVID-19, it will be understood that the pharmaceutically active compounds disclosed herein also have broad spectrum antiviral activity, anthelmintic activity, and anticancer activity.

The term “nanoparticle” or “nanoparticulate” is used herein to mean a particle having an average diameter of less than or equal to 1 micron (m), but greater than or equal to 1 nanometre (nm), i.e. in the range 1-1000 nm. These terms are clear and well understood by a person skilled in the art, without any confusion, not least as evidenced by Petros and DeSimone, Nature Reviews Drug Discovery, 2010, 9, 615-627.

Unless otherwise stated, the term “particle size”, “average diameter” and the like are used herein to refer to the z-average particle diameter (D_{z}), which may be determined by Dynamic Light Scattering.

Unless otherwise stated, the term polydispersity index (PdI), is in reference to the measurement provided by dynamic light scattering, in which perfect monodispersity is 0.

The term “consisting essentially of” is used herein to denote that a given product or method consists of only designated materials or steps and optionally other materials or steps that do not materially affect the characteristic(s) of the claimed invention. Suitably, a product which consists essentially of a designated material (or materials) comprises greater than or equal to 85% of the designated material, more suitably greater than or equal to 90%, more suitably greater than or equal to 95%, most suitably greater than or equal to 98% of the designated material(s).

Unless otherwise stated, the weight percentages (“wt %”) discussed herein relate to the % by weight of a particular constituent as a proportion of the total weight of the composition.

Unless otherwise stated, the weight/volume percentages (“w/v %”) discussed herein relate to the weight of the indicated material (in grams) per 100 mL of solvent.

It is to be appreciated that references to “preventing” or “prevention” relate to prophylactic treatment and includes preventing, limiting or delaying viral, helminth or protozoal infection following a patient's exposure to a virus, helminth or protozoa. This may involve preventing, limiting or delaying the appearance of clinical symptoms developing in a patient that may be afflicted with or exposed to the virus, helminth or protozoa but does not yet experience or display clinical or subclinical symptoms of the state, disorder or condition. Such prevention may prevent or reduce onward transmission of the virus, helminth or protozoa.

It will be further appreciated that references to “treatment” or “treating” of viral, helminth or protozoal infection includes: (1) inhibiting the symptoms of the infection, i.e., arresting, reducing or delaying the development of the disease or a relapse thereof (in case of maintenance treatment) or at least one clinical or subclinical symptom thereof; or (2) relieving or attenuating the infection, i.e. causing regression of the state, disorder or condition or at least one of its clinical or subclinical symptoms. Such treatment may prevent or reduce onward transmission of the virus, helminth or protozoa.

In the context of the invention, the terms “preventing” or “prevention” should not be considered to refer only to formulations which are completely effective in treating an infection, but also to cover formulations which are partially effective as well.

Moreover, when considered from the perspective of a population of patients for treatment, the terms “preventing” and “prevention” should be considered to cover formulations which are useful at reducing the rate of incidence of viral, helminth or protozoal infection in that target population, as well as medicaments which are useful at completely eradicating the viral, helminth or protozoal infection from that target population.

A “therapeutically effective amount” means the amount of pharmaceutically active compound that, when administered to a patient for treating and/or preventing a disease, is sufficient to effect such treatment/prevention for the viral, helminth or protozoal infection. The “therapeutically effective amount” will vary depending on the pharmaceutically active compound (e.g. niclosamide and/or nitazoxanide), the severity of the infection and the age, weight, etc., of the patient to be treated.

### Pharmaceutically Active Compounds

The present invention relates to formulations of pharmaceutically active compounds. In embodiments, the pharmaceutically active compounds are defined herein as compounds which exhibit a Cₘₐₓ/EC₉₀ ratio of greater than 1, as determined in Clin. Phamacol. Ther., 2020, DOI: 10.1002/cpt.1909, and may be selected from tipranavir, nitazoxanide, niclosamide, nelfinavir, remdesivir, favipiravir, eltrombopag, lopinavir, ritonavir, mefloquine, chloroquine, and anidulafungin. In preferred embodiments, the term “pharmaceutically active compound” is used herein to refer to compounds found to have a Cₘₐₓ/EC₉₀ (with respect to SARS-CoV-2) ratio in excess of 2, such as tipranavir, nitazoxanide, niclosamide, nelfinavir, remdesivir, favipiravir, and eltrombopag. The pharmaceutically active compounds may additionally, or alternatively, be members of the salicylanilide and thiazolide classes of drugs.

In embodiments, the pharmaceutically active compound is a member of the salicylanilide class of drugs or of the thiazolide class of drugs. In embodiments, the pharmaceutically active compound is a member of the salicylanilide class of drugs. In embodiments, the pharmaceutically active compound is a member of the thiazolide class of drugs. In embodiments, the pharmaceutically active compounds are selected from niclosamide and tizoxanide prodrugs, such as nitazoxanide. In certain embodiments, the pharmaceutically active compound is a tizoxanide prodrug, such as nitazoxanide. In yet other embodiments, the pharmaceutically active compound is niclosamide.

### Solid Compositions

The formulation may be in the form of a solid composition, wherein the pharmaceutically active compounds are present as nanoparticles dispersed within a solid matrix of carrier materials.

The nanoparticles of pharmaceutically active compound have an average particle size of less than or equal to 1 micron (m). Preferably, the nanoparticles of pharmaceutically active compound have an average particle size of between 100 and 1000 nm. Further preferably, the nanoparticles of pharmaceutically active compound have an average particle size between 300 and 950 nm. Yet further preferably, the nanoparticles of pharmaceutically active compound have an average particle size between 500 and 900 nm.

The polydispersity of the nanoparticles of pharmaceutically active compound may be less than or equal to 0.8, preferably less than or equal to 0.6, and more preferably less than or equal to 0.4.

The solid composition is preferably in the form of a free-flowing powder, but may alternatively be in a granular form or a tablet. The solid composition may comprise solid particles or granules of larger size, for example, 5 to 30 microns (m) in size, wherein each particle or granule contains a plurality of nanoparticles of pharmaceutically active compound dispersed within the carrier materials. These larger particles or granules disperse when the solid composition is mixed with an aqueous medium to release discrete nanoparticles of pharmaceutically active compound.

It is preferred that each nanoparticle within the solid composition comprises a single pharmaceutically active compound. However, in embodiments each nanoparticle may comprise a mixture of pharmaceutically active compounds. This may be achieved by formulating multiple pharmaceutically active compounds simultaneously.

The solid composition may include a single plurality of nanoparticles, each nanoparticle comprising a single pharmaceutically active compound. Alternatively, the solid composition may include multiple pluralities of nanoparticles, each plurality comprising a single pharmaceutically active compound. This may be achieved by formulating multiple pharmaceutically active compounds simultaneously to produce multiple pluralities of nanoparticles, or it may be achieved by commixing multiple solid compositions, each of which includes a single plurality of nanoparticles.

The carrier materials comprise at least one hydrophilic polymer and at least one sugar. Surprisingly, the combination of hydrophilic polymers and sugars was found to provide solid compositions in which the pharmaceutically active compounds were nanoparticulate in nature and the solid composition formed a free-flowing powder, without surface tackiness.

The following hydrophilic polymers are suitable for use in the present invention: polyvinyl alcohols, poloxamers, polyvinylpyrrolidones, hydroxypropyl celluloses, and hydroxypropyl methyl celluloses, for example polyvinyl alcohols, poloxamers, hydroxypropyl celluloses, and hydroxypropyl methyl celluloses. Preferred hydrophilic polymers are poloxamers and hydroxypropyl methyl celluloses.

In an embodiment, the hydrophilic polymer is polyvinyl alcohol. The polyvinyl alcohol may have a weight average molecular weight between 5000 and 200000 Da, suitably with a 75-90% hydrolysis level (i.e. % free hydroxyls). In a particular embodiment, the polyvinyl alcohol has a 75-90% hydrolysis level. In another embodiment, the polyvinyl alcohol has a 75-85% hydrolysis level. In a particular embodiment, the polyvinyl alcohol has a weight average molecular weight between 9000 and 10000 Da, suitably with an 80% hydrolysis level.

In an embodiment, the hydrophilic polymer is a poloxamer. A “poloxamer” is a non-ionic triblock copolymer comprising a central hydrophobic chain of polyoxypropylene, and hydrophilic chains of polyoxyethylene either side of this central hydrophobic chain. A “poloxamer” is typically named with the letter “P” followed by three numerical digits (e.g. P407), where the first two digits multiplied by 100 gives the approximate molecular mass of the polyoxypropylene chain, and the third digit multiplied by 10 provides the percentage polyoxyethylene content of the poloxamer. For example, P407 is a poloxamer having a polyoxypropylene molecular mass of about 4,000 g/mol and a polyoxyethylene content of about 70%. Poloxamers are also known as Pluronics®, as well as by several other commercial names. The poloxamer is suitably a pharmaceutically acceptable poloxamer. In a particular embodiment, the poloxamer is poloxamer P407.

In embodiments, the polyvinylpyrrolidone has a weight average molecular weight of 1000 to 1,000,000 g/mol. In a particular embodiment, the polyvinylpyrrolidone has a weight average molecular weight of 1000 to 40000 g/mol, preferably 2000 to 20000 g/mol. In embodiments, the polyvinylpyrrolidone has a K value between 5 and 30, preferably between 10 and 20, most preferably between 12 and 17. Alternatively the K value may be about 12, about 15, or about 17. As is known in the art, K value is derived from relative viscosity measurements and calculated according to Fikentscher's equation.

In an embodiment, the hydrophilic polymer is a HPMC. In a particular embodiment, the HPMC has a weight average molecular weight of 10000 to 400000 Da. In a particular embodiment, the HPMC has a weight average molecular weight of about 10000 Da.

Generally, any naturally occurring monosaccharide, disaccharide, and oligosaccharide may be suitable in the solid composition of the present invention. Disaccharides are defined as carbohydrates consisting of two monosaccharide residues. Oligosaccharides are defined herein as carbohydrates consisting of between 3 and 10 monosaccharide residues.

Monosaccharides may be selected from ribose, arabinose, xylose, lyxose, ribulose, xylulose, allose, altrose, glucose, mannose, gulose, idose, galactose, talose, psicose, fructose, sorbose, and tagatose. Either of the D- or L-isomers may be used, with the naturally occurring isomer being preferred.

Disaccharides may be selected from any binary combination of the above monosaccharides. Preferred disaccharides are lactose and sucrose.

Oligosaccharides may be selected from any combination of the above monosaccharides.

### Particular Formulations of the Solid Composition

In a particular embodiment, the solid composition comprises up to 80 wt % pharmaceutically active compound. In a particular embodiment, the solid composition comprises up to 70 wt % pharmaceutically active compound. In another embodiment, the solid composition comprises up to 60 wt % pharmaceutically active compound. In another embodiment, the solid composition comprises up to 50 wt % pharmaceutically active compound. In another embodiment, the solid composition comprises 30 to 80 wt % pharmaceutically active compound. In another embodiment, the solid composition comprises 40 to 70 wt % pharmaceutically active compound. In another embodiment, the solid composition comprises 50 to 60 wt % pharmaceutically active compound. In certain embodiments, the remainder of the composition consists of the hydrophilic polymer and the sugar.

Suitably, the solid composition comprises 10 to 60 wt % of the hydrophilic polymer and sugar combined, more suitably 20 to 60 wt %, even more suitably 25 to 50 wt %, most suitably 25 to 40 wt %. In a particular embodiment, the solid composition comprises 25 to 35 wt % of the hydrophilic polymer and sugar combined.

In a particular embodiment, the solid composition comprises 5 to 50 wt % of hydrophilic polymer. In another embodiment, the solid composition comprises 10 to 30 wt % of hydrophilic polymer. In another embodiment, the solid composition comprises 15 to 30 wt % of hydrophilic polymer. In a particular embodiment, the solid composition comprises 25 wt % of hydrophilic polymer.

In a particular embodiment, the solid composition comprises 5 to 50 wt % of sugar. In another embodiment, the solid composition comprises 10 to 30 wt % of sugar. In another embodiment, the solid composition comprises 15 to 30 wt % of sugar. In a particular embodiment, the solid composition comprises 25 wt % of sugar.

In an embodiment, the solid composition comprises the hydrophilic polymer and sugar in a weight ratio of between 3:1 and 1:3. In a particular embodiment, the solid composition comprises the hydrophilic polymer and sugar in a weight ratio of between 2:1 and 1:2. In another embodiment, the solid composition comprises the hydrophilic polymer and sugar in a weight ratio of about 1:1.

The solid composition may comprise:
10 to 80 wt % pharmaceutically active compound,
5 to 45 wt % hydrophilic polymer, and
5 to 45 wt % sugar.

The solid composition may comprise:
20 to 70 wt % pharmaceutically active compound,
5 to 40 wt % hydrophilic polymer, and
5 to 40 wt % sugar.

The solid composition may comprise:
50 to 60 wt % pharmaceutically active compound,
10 to 30 wt % hydrophilic polymer, and
10 to 30 wt % sugar.

In one embodiment, the pharmaceutically active compound is a thiazolide, preferably a tizoxanide prodrug such as nitazoxanide, the hydrophilic polymer is a poloxamer, and the sugar is at least one of lactose and sucrose.

In a preferred embodiment, the pharmaceutically active compound is a thiazolide, preferably a tizoxanide prodrug such as nitazoxanide, the hydrophilic polymer is a poloxamer, and the sugar is lactose. The composition may comprise 40 to 60 wt % thiazolide, 10 to 40 wt % poloxamer, and 10 to 40 wt % lactose. The composition may comprise 50 wt % thiazolide, 20 to 30 wt % poloxamer, and 20 to 30 wt % lactose. The composition may comprise 60 wt % thiazolide, 30 wt % poloxamer and 10 wt % lactose.

In a preferred embodiment, the pharmaceutically active compound is a thiazolide, preferably a tizoxanide prodrug such as nitazoxanide, the hydrophilic polymer is a poloxamer, and the sugar is sucrose. The composition may comprise 20 to 60 wt % thiazolide, 10 to 40 wt % poloxamer, and 10 to 40 wt % sucrose. The composition may comprise 60 wt % thiazolide, 10 to 30 wt % poloxamer, and 10 to 30 wt % sucrose. The composition may comprise 60 wt % thiazolide, 20 wt % poloxamer, and 20 wt % sucrose.

In a preferred embodiment, the pharmaceutically active compound is a salicylanilide, such as niclosamide, the hydrophilic polymer is a hydroxypropyl methyl cellulose, and the sugar is sucrose. The composition may comprise 20 to 60 wt % salicylanilide, 15 to 30 wt % hydroxypropyl methyl cellulose, and 15 to 30 wt % sucrose. The composition may comprise 50 to 60 wt % salicylanilide, 20 to 25 wt % hydroxypropyl methyl cellulose, and 20 to 25 wt % sucrose. The composition may comprise 50 wt % salicylanilide, 25 wt % hydroxypropyl methyl cellulose, and 25 wt % sucrose. The composition may comprise 60 wt % salicylanilide, 20 wt % hydroxypropyl methyl cellulose, and 20 wt % sucrose.

In a preferred embodiment, the pharmaceutically active compound is niclosamide, the hydrophilic polymer is a polyvinylpyrrolidone, and the sugar is sucrose. The composition may comprise 20 to 60 wt % niclosamide, 15 to 30 wt % polyvinylpyrrolidone, and 15 to 30 wt % sucrose. The composition may comprise 50 to 60 wt % niclosamide, 20 to 25 wt % polyvinylpyrrolidone, and 20 to 25 wt % sucrose. The composition may comprise 50 wt % niclosamide, 25 wt % polyvinylpyrrolidone, and 25 wt % sucrose. The composition may comprise 60 wt % niclosamide, 20 wt % polyvinylpyrrolidone, and 20 wt % sucrose.

In a preferred embodiment, the pharmaceutically active compound is niclosamide, the hydrophilic polymer is a polyvinylpyrrolidone, and the sugar is lactose. The composition may comprise 20 to 60 wt % niclosamide, 15 to 30 wt % polyvinylpyrrolidone, and 15 to 30 wt % lactose. The composition may comprise 50 to 60 wt % niclosamide, 20 to 25 wt % polyvinylpyrrolidone, and 20 to 25 wt % lactose. The composition may comprise 50 wt % niclosamide, 25 wt % polyvinylpyrrolidone, and 25 wt % lactose. The composition may comprise 60 wt % niclosamide, 20 wt % polyvinylpyrrolidone, and 20 wt % lactose.

It will be understood that, in any of the above solid compositions, the solid composition may consist essentially of, or consist of, the indicated amounts of pharmaceutically active compound, hydrophilic polymer, and sugar.

### General Process for Preparing the Solid Composition

The general procedure for the preparation of the solid composition is as follows:
(a) providing an active solution comprising the pharmaceutically active compound in a water-miscible solvent;
(b) providing a carrier material solution comprising one or more hydrophilic polymers and one or more sugars in an aqueous solvent;
(c) mixing the solutions prepared in steps (a) and (b); and
(d) removing the mixed solvent to produce the solid composition.

The solutions are typically provided by dissolving the pharmaceutically active compound in the water-miscible solvent and by dissolving the one or more hydrophilic polymers and one or more sugars in the aqueous solvent. The pharmaceutically active compound, hydrophilic polymers and sugars are drawn from those described for the solid composition.

Any water-miscible solvent that is capable of dissolving a given pharmaceutically active compound in the required concentrations may be used to process it. Suitable water-miscible solvents are acetone, butanol, dimethylsulfoxide (DMSO), dimethylformamide (DMF), ethanol, methanol, propanol and mixtures thereof. Particularly suitable solvents are DMSO and acetone/ethanol mixtures. Acetone/ethanol mixtures may be in any suitable ratio. Preferred volume ratios are in the range of 90/10 to 50/50, in the range of 85/15 to 70/30. A most preferred volume ratio is 80/20. The pharmaceutically active compound may be present in a concentration of at least 5 w/v %. Preferably the pharmaceutically active compound is present in a concentration in the range of 6 to 30 w/v %.

The aqueous solvent is typically deionised water. The carrier material may be present in the carrier material solution in a concentration up to 5 w/v %, preferably in a concentration in the range of 1 to 4 w/v %, most preferably in the range of 2 to 3 w/v %.

The active solution and the carrier material solution are mixed in a volume ratio in the range of 1:15 and 1:1, preferably in the range of 1:9 and 3:4. Preferably the volume ratio is in the range of 1:9 to 1:2.

After mixing, the total solids content (i.e. the sum of the pharmaceutically active compound and the carrier materials in the mixed solvent) may be in the range of 1 to 10 w/v %, preferably 2 to 8 w/v %, further preferably 3 to 7 w/v %, most preferably 4 to 6 w/v %. Alternatively, the total solids content may be about 5 w/v %.

The solutions may be mixed by any suitable method. Typically, a rotary stirring system is used, such as a magnetic or overhead stirrer. The pharmaceutically active compound has a reduced solubility in the mixed solvent system, resulting in a supersaturated solution. The pharmaceutically active compound consequently precipitates from the solution, producing nanoparticles which are stabilised by the carrier materials. The mixing may be instantaneous, or it may take place over a time period. The latter may be achieved through the use of a pump, such as a peristaltic pump, operating at a rate of 1 to 20 mL/min, preferably at a rate of about 5 mL/min.

It may be beneficial to homogenise the mixed solutions to reduce the incidence of aggregation and promote homogeneity in particle size. Any suitable homogeniser may be used, such as a rotary homogeniser. Similarly, it may be beneficial to sonicate the mixed solutions. Any suitable sonicator may be used, such as a probe sonicator.

In some embodiments, the active solution is heated to increase the solubility of the pharmaceutically active compound therein, allowing higher concentrations to be used and increasing the degree of supersaturation on mixing with the carrier material solution, particularly as the carrier material solution is maintained at ambient temperature (approximately 25° C.). Any suitable temperature may be used, for example 30 to 90° C., 40 to 80° C., 50 to 70° C., or about 60° C.

Any suitable method of removing the mixed solvent may be used, on the condition that it does not provide the nanoparticles with the opportunity to aggregate. This may be achieved by either removing the solvent extremely rapidly, or by rapidly solidifying the dispersion and subliming the solid solvent (e.g. lyophilisation). The former method is preferred, utilising spray-drying or spray-granulating techniques, due to their high throughput and acceptability in pharmaceutical applications. It will be understood that the parameters of spray-drying and spray-granulation processes, such as flow rate and temperature, may be varied to achieve effective drying and attain the desired powdery and granular products. If required, the resulting solid may be subjected to further drying procedures, such as being dried in vacuo, to remove any residual solvents.

### Particular Processes for Preparing Solid Compositions Comprising a Thiazolide

DMSO has been found to be a particularly suitable water-miscible solvent for thiazolides, especially tizoxanide prodrugs, such as nitazoxanide, with the concentration of thiazolide in the thiazolide solution preferably being in the range of 20 to 40 w/v %, more preferably being about 30 w/v %. It is preferable to maintain the thiazolide solution at an elevated temperature. By elevated temperature it is meant a temperature between ambient and the boiling point of the solvent. Such elevated temperatures may be in the range of 30 to 90° C., 40 to 80° C., 50 to 70° C., or about 60° C.

A preferred hydrophilic polymer for producing solid compositions comprising a thiazolide, especially tizoxanide prodrugs such as nitazoxanide, is poloxamer, such as poloxamer 407. Preferred sugars for producing such solid compositions are sucrose and lactose. Preferred concentrations for the carrier materials in the carrier material solutions are in the range of 1 to 5 w/v %, preferably 2 to 3 w/v %. The weight ratio of poloxamer to sugar may be in the range of 3:1 to 1:3. Where the sugar is lactose, suitable weight ratios of poloxamer to sugar may be in the range of 2:3 to 3:2, or be about 3:1. Where the sugar is sucrose, suitable weight ratios of poloxamer to sugar may be in the range of 3:1 to 1:3.

The thiazolide solution and carrier material solution may be mixed in a volume ratio of about 1:9. In certain embodiments, after mixing, the thiazolide and carrier materials may be present in the mixed solution in a weight ratio in the range of 50:50 to 60:40. Particular weight ratios of thiazolide to poloxamer to sucrose that may be suitable in the mixed solution are 60:30:10, 60:20:20, or 60:10:30. Particular weight ratios of thiazolide to poloxamer to lactose that may be suitable in the mixed solution are 50:30:20, 50:25:25, 50:20:30, or 60:30:10. In the foregoing compositions, tizoxanide prodrugs (such as nitazoxanide) are preferred thiazolides.

In a first exemplary embodiment, the method comprises the following steps:
(a) providing a thiazolide solution comprising the thiazolide dissolved in DMSO;
(b) providing a carrier material solution comprising poloxamer and one of lactose or sucrose dissolved in an aqueous solvent;
(c) mixing the solutions prepared in steps (a) and (b); and
(d) removing the mixed solvent to produce the solid composition.

In a second exemplary embodiment, the method comprises the following steps:
(a) providing a thiazolide solution comprising the thiazolide dissolved in DMSO;
(b) providing a carrier material solution comprising poloxamer and lactose dissolved in an aqueous solvent;
(c) mixing the solutions prepared in steps (a) and (b); and
(d) removing the mixed solvent to produce the solid composition.

In a third exemplary embodiment, the method comprises the following steps:
(a) providing a thiazolide solution comprising the thiazolide dissolved in DMSO;
(b) providing a carrier material solution comprising poloxamer and sucrose dissolved in an aqueous solvent;
(c) mixing the solutions prepared in steps (a) and (b); and
(d) removing the mixed solvent to produce the solid composition.

Preferred thiazolides for the above exemplary embodiments are tizoxanide prodrugs, such as nitazoxanide. In variations of the above exemplary embodiments, the thiazolide solution may be heated, mixing the solutions may additionally comprise homogenisation, and/or removing the solvent may comprise spray-drying.

Particular Processes for Preparing Salicylanilide Containing Solid Compositions Acetone/ethanol and butanone/ethanol mixtures (for example acetone/ethanol mixtures) have been found to be particularly suitable water-miscible solvents for salicylanilides, such as niclosamide, preferably the volume ratio of acetone (or butanone) to ethanol is in the range of 90/10 to 50/50, or in the range of 85/15 to 70/30. A most preferred volume ratio is 80/20. The concentration of salicylanilide in the salicylanilide solution preferably being in the range of 1 to 20 w/v %, more preferably being in the range of 2 to 10 w/v %, most preferably being in the range of 4 to 8 w/v %. A most preferable concentration is about 6 w/v %. It is preferable to maintain the salicylanilide solution at an elevated temperature, such as about 60° C.

A preferred hydrophilic polymer for producing salicylanilide solid compositions is hydroxypropyl methyl cellulose. A preferred sugar for producing salicylanilide solid compositions is sucrose. Preferred concentrations for the carrier materials in the carrier material solutions are in the range of 1 to 5 w/v %, preferably 2 to 3 w/v %, or about 3 w/v %. The weight ratio of hydroxypropyl methyl cellulose to sucrose may be in the range of 3:1 to 1:3. Preferred weight ratios of hydroxypropyl methyl cellulose to sucrose may be about 1:1.

The carrier material solution and salicylanilide solution may be mixed in a volume ratio in the range of 2:1 to 4:3. In certain embodiments, after mixing, the salicylanilide and carrier materials may be present in the mixed solution in a weight ratio in the range of 50:50 to 70:30. A particular weight ratio of niclosamide to hydroxypropyl methyl cellulose to sucrose that may be suitable in the mixed solution is 66:17:17, 60:20:20, or 50:25:25.

In an exemplary embodiment, the method comprises the following steps:
(a) providing a salicylanilide solution comprising salicylanilide dissolved in a mixture of acetone and ethanol;
(b) providing a carrier material solution comprising hydroxypropyl methyl cellulose and sucrose dissolved in an aqueous solvent;
(c) mixing the solutions prepared in steps (a) and (b); and
(d) removing the mixed solvent to produce the solid composition.

A preferred salicylanilide for use in the above method is niclosamide. In variations of the above exemplary embodiment, the mixture of acetone and ethanol may be in a volume ratio of about 80:20, the salicylanilide solution may be heated (e.g. to about 60° C.), mixing the solutions may additionally comprise sonication, and/or removing the solvent may comprise spray-drying.

A further preferred hydrophilic polymer for producing salicylanilide solid compositions is polyvinylpyrrolidone. Preferred sugars for producing salicylanilide solid compositions are sucrose and lactose. Preferred concentrations for the carrier materials in the carrier material solutions are in the range of 1 to 5 w/v %, preferably 2 to 3 w/v %, or about 3 w/v %. The weight ratio of polyvinylpyrrolidone to sugar may be in the range of 3:1 to 1:3. Preferred weight ratios of polyvinylpyrrolidone to sugar may be about 1:1.

The carrier material solution and salicylanilide solution may be mixed in a volume ratio in the range of 2:1 to 4:3. In certain embodiments, after mixing, the salicylanilide and carrier materials may be present in the mixed solution in a weight ratio in the range of 50:50 to 70:30. A particular weight ratio of niclosamide to polyvinylpyrrolidone to sugar that may be suitable in the mixed solution is 66:17:17, 60:20:20, or 50:25:25.

In an exemplary embodiment, the method comprises the following steps:
(a) providing a salicylanilide solution comprising salicylanilide dissolved in a mixture of acetone and ethanol or a mixture of butanone and ethanol;
(b) providing a carrier material solution comprising polyvinylpyrrolidone and sugar (either lactose or sucrose) dissolved in an aqueous solvent;
(c) mixing the solutions prepared in steps (a) and (b); and
(d) removing the mixed solvent to produce the solid composition.

A preferred salicylanilide for use in the above method is niclosamide. In variations of the above exemplary embodiment, the mixture of acetone and ethanol, or butanone and ethanol, may be in a volume ratio of about 80:20, the salicylanilide solution may be heated (e.g. to about 60° C.), mixing the solutions may additionally comprise sonication, and/or removing the solvent may comprise spray-drying.

### Pharmaceutical Compositions Comprising the Solid Composition

The present invention provides a pharmaceutical composition comprising any of the aforementioned solid compositions. The pharmaceutical compositions of the present invention may further comprise one or more additional pharmaceutically acceptable excipients.

The solid compositions of the invention may be formulated into a form suitable for oral use (for example as tablets, lozenges, hard or soft capsules, or dispersible powders or granules) by techniques known in the art. As such, the solid compositions of the invention may be mixed with one or more additional pharmaceutical excipients during this process, such as antiadherants, binders, coatings, enterics, disintegrants, fillers, diluents, flavours, colours, lubricants, glidants, preservatives, sorbents, and sweeteners.

A preferred pharmaceutical composition comprising the solid compositions defined herein is an inhalable dry powder. Such a powder permits delivery of the pharmaceutically active compounds directly to the respiratory tract, particularly the lower respiratory tract, by a dry powder inhaler. It is preferred that the solid composition is in the form of particles of 1 to 20 μm in size, preferably 1 to 5 μm in size. It will be understood that particles within this size range are most effectively delivered to the peripheral airways and that the size may be tailored depending on how far the particles are required to penetrate into the respiratory tract. On contact with the wet surfaces of the airways, the hydrophilic polymer and sugar dissolve, exposing the nanoparticles of pharmaceutically active compounds directly to the tissues likely to be the site of infection. On exposure, the nanoparticles release the pharmaceutically active compound into solution and/or are directly taken up by cells. Lactose may be used as an excipient in inhalable dry powder formulations, specifically as a bulking agent and carrier.

A preferred pharmaceutical composition comprising the solid compositions defined herein is a suspension of the solid composition in a volatile propellant. Such a suspension permits delivery of the pharmaceutically active compounds directly to the respiratory tract by a pressurised metered-dose inhaler.

### Therapeutic Uses of the Solid Composition and Pharmaceutical Compositions Comprising Same

The present invention provides the aforementioned solid compositions, or the pharmaceutical compositions comprising them, for use as medicaments.

In particular, the present invention provides the aforementioned solid compositions, or the pharmaceutical compositions comprising them, for use in treating and/or preventing a viral infection, helminth infection, or protozoal infection, optionally wherein the viral infection is a coronavirus infection, such as SARS-CoV-2 infection.

The present invention also provides a method of treating and/or preventing a viral infection, helminth infection, or protozoal infection, optionally wherein the viral infection is a coronavirus infection, such as SARS-CoV-2 infection, the method comprising administering a therapeutically effective amount of the aforementioned solid compositions or pharmaceutical compositions, to a patient suffering from, or at risk of suffering from, the viral infection.

The use or administration of the solid composition or pharmaceutical composition may be via an intranasal or pulmonary route, such as via a dry powder inhaler or pressurised metered-dose inhaler.

The use or administration of the solid composition or pharmaceutical composition may be via a transdermal route, using a transdermal patch.

The present invention further provides a use of the aforementioned solid compositions or pharmaceutical compositions in the manufacture of a medicament for use in the treatment and/or prevention of a viral infection, helminth infection, or protozoal infection, optionally wherein the viral infection is a coronavirus infection, such as SARS-CoV-2 infection.

### Aqueous Dispersions

The present invention provides an aqueous dispersion, comprising a plurality of nanoparticles of pharmaceutically active compound dispersed in an aqueous medium, wherein the pharmaceutically active compound is selected from tizoxanide prodrugs, such as nitazoxanide, and niclosamide, each nanoparticle being stabilised by the one or more hydrophilic polymers and/or the one or more sugars adsorbed to the surface of the nanoparticle.

The aqueous dispersion may be, obtainable by, obtained by, or directly obtained by dispersing the aforementioned solid composition in an aqueous medium. Suitably, an aqueous dispersion is prepared immediately prior to use.

Each nanoparticle within the aqueous dispersion may comprise a single pharmaceutically active compound. However, in embodiments each nanoparticle may comprise a mixture of pharmaceutically active compounds. This may be achieved by formulating multiple pharmaceutically active compounds simultaneously.

The aqueous dispersion may include a single plurality of nanoparticles, each nanoparticle comprising a single pharmaceutically active compound. Alternatively, the aqueous dispersion may include multiple pluralities of nanoparticles, each plurality comprising a single pharmaceutically active compound. This may be achieved by dispersing more than one of the aforementioned solid compositions in the same aqueous medium. For example, a solid composition comprising only nitazoxanide nanoparticles and a solid composition comprising only niclosamide nanoparticles (each composition further comprising suitable hydrophilic polymers and sugars) may be dissolved in the same aqueous medium to provide an aqueous dispersion of nitazoxanide nanoparticles and niclosamide nanoparticles.

When the solid composition is dispersed in the aqueous medium, the hydrophilic polymer and/or sugar is dissolved within the aqueous medium to release the nanoparticles comprising the pharmaceutically active compound in a dispersed form. The nanoparticles of pharmaceutically active compound, which were formerly dispersed within a solid mixture of the hydrophilic polymer and sugar, then become dispersed within the aqueous medium in nanoparticulate form. The association of the hydrophilic polymer(s) and sugar(s) with the pharmaceutically active compound in the nanoparticles may impart stability to the nanoparticles, thereby preventing premature coagulation and aggregation.

The aqueous medium may be water. It is preferred that the aqueous medium is a saline solution, preferably 0.9 wt % saline (i.e. 0.9 wt % NaCl dissolved in water). Alternatively the aqueous medium may be phosphate buffered saline. The aqueous medium may comprise one of more acceptable diluents or excipients.

Suitably the relative amounts (including ratios) of pharmaceutically active compound, hydrophilic polymer(s), and sugar(s) are the same as defined above in relation to the solid composition. However, the skilled person will readily appreciate that their respective wt % values in the aqueous dispersion as a whole must be adjusted to take account of the aqueous medium. In a particular embodiment, the aqueous dispersion comprises 1 to 10 mg/mL total solids (i.e. the total mass of the pharmaceutically active compound, hydrophilic polymer, and sugar per mL of the aqueous dispersion is 1 to 10 mg).

Aqueous dispersions of the present invention are advantageously stable for prolonged periods, both in terms of chemical stability and the stability of the particles themselves (i.e. with respect to aggregation, coagulation, etc.).

Aqueous dispersions of the present invention allow a measured aliquot to be taken therefrom for accurate dosing in a personalised medicine regime.

The particle diameter and polydispersity of the nanoparticles comprising pharmaceutically active compound in the aqueous dispersion is as defined hereinbefore in relation to the solid composition. It will of course be appreciated that the particle diameter and polydispersity of the nanoparticles comprising pharmaceutically active compound present in the solid composition are measured by dispersing the solid composition in an aqueous medium to thereby form an aqueous dispersion of the present invention.

In an embodiment, the aqueous dispersion comprises a single hydrophilic polymer and a single sugar selected from those listed herein. In an alternative embodiment, the aqueous dispersion comprises two or more hydrophilic polymers and/or two or more sugars selected from those listed herein.

### Pharmaceutical Compositions Comprising the Aqueous Dispersion

The present invention provides a pharmaceutical composition comprising any of the aforementioned aqueous dispersions. The pharmaceutical compositions of the present invention may further comprise one or more additional pharmaceutically acceptable excipients.

The aqueous dispersion of the present invention may be administered as it is or further formulated with one or more additional excipients to provide a dispersion, elixir or syrup that is suitable for oral use, a dispersion that is suitable for parenteral administration (for example, a sterile aqueous dispersion for intravenous, subcutaneous, intramuscular, intraperitoneal, transdermal or intramuscular dosing), or a dispersion that is suitable for pulmonary use via a nebuliser (for example, a saline based aqueous dispersion).

In a particular embodiment, the pharmaceutical composition is an aqueous dispersion as described herein. Such dispersed formulations can be used to accurately measure smaller dosages, such as those suitable for administration to children.

In a particular embodiment, the pharmaceutical composition is in a form suitable for parenteral delivery, whether via intravenous or intramuscular delivery. In an alternative embodiment, the pharmaceutical composition is in a form suitable for pulmonary and/or intranasal delivery via a nebuliser. Delivery via nebuliser is particularly beneficial in the case of treating and/or preventing SARS-CoV-2 infection as it directs the pharmaceutically active compounds directly to the affected tissues of the upper and lower respiratory tract, including the intranasal sinus. In addition, it is non-invasive and simple to administer.

It will be appreciated that different pharmaceutical compositions of the invention may be obtained by conventional procedures, using conventional pharmaceutical excipients, well known in the art.

The pharmaceutical compositions of the invention contain a therapeutically effective amount of active. A person skilled in the art will know how to determine and select an appropriate therapeutically effective amount of active to include in the pharmaceutical compositions of the invention.

### Therapeutic Uses of the Aqueous Dispersions

The present invention provides the aforementioned aqueous dispersions, or the pharmaceutical compositions comprising them, for use as medicaments.

In particular, the present invention provides the aforementioned aqueous dispersions, or the pharmaceutical compositions comprising them, for use in treating and/or preventing a viral infection, helminth infection, or protozoal infection, optionally wherein the viral infection is a coronavirus infection, such as SARS-CoV-2 infection.

The present invention also provides a method of treating and/or preventing a viral infection, helminth infection, or protozoal infection, optionally wherein the viral infection is a coronavirus infection, such as SARS-CoV-2 infection, the method comprising administering a therapeutically effective amount of the aforementioned aqueous dispersions or pharmaceutical compositions, to a patient suffering from, or at risk of suffering from, the viral infection.

The use or administration of the aqueous dispersion or pharmaceutical composition may be via an intranasal or pulmonary route, such as via a nebuliser. Alternatively, the aqueous dispersion or pharmaceutical composition may be used or administered orally or parenterally (e.g. intramuscularly or subcutaneously).

The present invention further provides a use of the aforementioned aqueous dispersions or pharmaceutical compositions in the manufacture of a medicament for use in the treatment and/or prevention of a viral infection, helminth infection, or protozoal infection, optionally wherein the viral infection is a coronavirus infection, such as SARS-CoV-2 infection.

### Injectable Formulations of Active Compound

The present invention also provides an intramuscularly-injectable formulation or a subcutaneously-injectable formulation of nanoparticles of pharmaceutically active compound comprising the aforementioned aqueous dispersions, or the pharmaceutical compositions comprising said aqueous dispersions.

Said formulations may be in substantially solid form (e.g. a paste) or liquid form, in which the pharmaceutically active compound is present in the form of nanoparticles. The nanoparticles of pharmaceutically active compound may be dispersed within at least one hydrophilic polymer and at least one sugar. When in liquid form, each nanoparticle of pharmaceutically active compound may be provided as a core around which an outer layer composed of the at least one hydrophilic polymer and at least one sugar is provided.

The injectable formulations of nanoparticles of pharmaceutically active compound are advantageously designed for administration as a depot injection, so as to provide prophylaxis and/or treatment, especially in respect of SARS-CoV-2 infection.

Preferably, the injectable formulation of nanoparticles of pharmaceutically active compound provides a controlled release bolus formulation of pharmaceutically active compound, which, when administered to a patient (via intramuscular or subcutaneous injection), releases the pharmaceutically active compound into the bloodstream of the patient over a period of at least about two weeks from the date of administration. Further preferably the period of release is at least about three weeks, yet further preferably at least about one month, more preferably at least about three months, and most preferably at least about six months, from the date of administration of the injection.

### EXAMPLES

### General Procedure for the Measurement of Particle Sizes

A sample of the solid composition was dissolved in saline (0.9% w/v) at a concentration of 1 mg/mL and immediately analysed by Dynamic Light Scattering (DLS) to determine its z-average hydrodynamic diameter (D_{z}). These measurements were performed in triplicate and the average reported. The specific instrument used was a Malvern® Panalytical® ZetaSizer® Ultra Photon Correlation Spectroscope, the instrument being set to record the intensity using backscattering detector at a temperature of 25° C., with a fluorescent sample filter in place. Data analysis was conducted using the general-purpose model within the ZS Xplorer software.

### Materials

Polymers, surfactant and sugars were purchased from Merck unless otherwise stated, Nitazoxanide and niclosamide were purchased from Biosynth Carbosynth. All materials were used as supplied with no further purification. The weight average molecular weights of the polymers are as follows; Hydroxypropyl methyl cellulose 10,000, hydroxypropyl cellulose 80,000, polyvinyl alcohol (80% hydrolysed) 9,000-10,000. Sucrose and sodium dodecyl sulphate were used at a purity of >99.5% and >99.0% respectively. Nitazoxanide and niclosamide were supplied at a minimum purity of 99% and 98% respectively.

### Comparative Example 1: Unsuccessful Nitazoxanide Formulations

Nitazoxanide was screened against carrier materials selected from excipients used for pulmonary administration of FDA-approved medicines, namely: polyvinylalcohol (PVA), Pluronic® F-127 (F127, also known as Poloxamer 407), hydroxypropyl cellulose (HPC), Tween 20 (polysorbate 20), Tween 80 (polysorbate 80) and sodium dodecyl sulfate (SDS).

The solid compositions were obtained by flash nanoprecipitation, involving rapid addition of a nitazoxanide-DMSO solution into an aqueous solution of the carrier materials to generate suspensions with a 17.5% v/v DMSO content. The total solid content (i.e. the total content of nitazoxanide and carrier materials in the mixed solution) was 5 w/v %. These suspensions were immediately spray dried to give powdered products with typical yields of 35%.

As a specific example, the nitazoxanide/F127/Tween 20 (70/20/10 wt %) formulation was produced as follows. 100 mg of F127 and 50 mg of Tween 20 were dissolved in 8.25 mL of water to form the carrier material solution. Separately, 350 mg of nitazoxanide was dissolved in 1.75 mL of anhydrous DMSO and rapidly added to the carrier material solution under stirring. The suspension was immediately passed through a spray dryer at a flow-rate of 5 mL/minute (Buchi Mini B-290; Aspirator 100%, Nitrogen (cylinder pressure 5 bar) Q-flow gauge 45, Outlet temperature 65° C.). The powder was collected and residual water was removed by freeze drying (Virtis Benchtop Pro).

The particle size of each formulation was assessed by DLS as outlined in the general method above. The results are outline in Table 1 (below).

TABLE 1

| Nitazoxanide/Polymer/Surfactant | |
|---|---|
| (70/20/10 wt %) Formulation | DLS Analysis |

| Polymer | Surfactant | D_{z} ± σ (nm) | PdI |
|---|---|---|---|
| PVA | Tween 20 | — | — |
| | Tween 80 | — | — |
| | SDS | 1980 ± 200 | 0.290 |
| F127 | Tween 20 | 1390 ± 80 | 0.396 |
| | Tween 80 | 1500 ± 300 | 0.406 |
| | SDS | 1690 ± 60 | 0.656 |
| HPC | Tween 20 | — | — |
| | Tween 80 | — | — |
| | SDS | — | — |

The majority of the formulations failed to produce formulations which could provide meaningful data. However, the samples using F127 did form microparticles.

Further samples were produced with a reduced ratio of DMSO to water (to 10 v/v %), in order to achieve a higher degree of supersaturation and particle nucleation, and a reduced nitazoxanide content of 50 wt %, in order to increase the relative content of carrier materials for covering the drug particle surfaces, limiting their growth. The solids content was maintained at 5 w/v %. F127 and Tween 20 ratios of 40:10, 30:20, 20:30, and 10:40 were tested. None of these formulations were effective, producing aggregates.

### Example 1: Development of Nitazoxanide Formulations

It was discovered that the incorporation of lactose into the formulations improved the quality of the dispersions formed from the formulations and additionally reduced the particle sizes. The results revealed a general increase in D_{z} values from 800 nm to 1820 nm as the weight composition of F127 decreased from 30 wt. % to 0 wt. %, highlighting the importance of the poloxamer in arresting particle growth and stabilising the particles within the aqueous dispersions. The results also identified three formulation candidates that met the product criteria of fine powders that readily dispersed in saline to yield nanoparticles with D_{z} values <1 μm. These were F127/Tween 20/lactose formulations with weight compositions (30/10/10 wt %), (20/10/20 wt %) and (20/0/30 wt %) that had D_{z} values of 800 nm, 930 nm and 860 nm, respectively. Of these three candidates, the (20/0/30) formulation was the most promising (Table 2), which suggested that Tween 20 had little effect on determining the success of the formulation outcome.

TABLE 2

| Formulation Composition (wt. %) | | | | DLS Analysis | |
|---|---|---|---|---|---|
| Nitazoxanide | F127 | Tween 20 | Lactose | D_{z} ± σ (nm) | PdI |
| 50 | 30 | 10 | 10 | 800 ± 20 | 0.413 |
| 50 | 20 | 10 | 20 | 930 ± 40 | 0.488 |
| 50 | 20 | 0 | 30 | 860 ± 30 | 0.282 |
| 50 | 10 | 30 | 10 | 1160 ± 10 | 0.396 |
| 50 | 10 | 20 | 20 | 1340 ± 60 | 0.438 |
| 50 | 10 | 0 | 40 | 1280 ± 100 | 0.548 |
| 50 | 0 | 10 | 40 | 1820 ± 400 | 0.583 |

### Example 2: Nitazoxanide Formulations

Further formulations were produced in the absence of Tween 20, with nitazoxanide contents of 50 wt % and varying F127 and lactose contents. All of the 50 wt % nitazoxanide formulations produced high quality nanodispersions, prompting attempts at higher nitazoxanide loadings. At the higher loading of 60 wt %, all of the formulations produced acceptable dispersions, with one formulation (nitazoxanide/F127/lactose 60/30/10 wt %) producing a nanodispersion, as shown in Table 3.

TABLE 3

| | |
|---|---|
| Formulation Composition (wt %) | DLS Analysis |

| Nitazoxanide | F127 | Lactose | D_{z} ± σ (nm) | PdI |
|---|---|---|---|---|
| 50 | 30 | 20 | 900 ± 50 | 0.437 |
| | 25 | 25 | 850 ± 5 | 0.372 |
| | 20 | 30 | 860 ± 30 | 0.282 |
| 60 | 30 | 10 | 850 ± 10 | 0.377 |
| | 25 | 15 | 1080 ± 130 | 0.471 |
| | 20 | 20 | 1030 ± 70 | 0.519 |
| | 15 | 25 | 1090 ± 80 | 0.520 |

To test the effect of the sugar, an alternative disaccharide, sucrose, was tested at the higher 60 wt % loading of nitazoxanide. All three formulations yielded fine powders that were readily dispersed in saline. Analysis by DLS revealed that sucrose had a positive impact on the formulation outcome compared to those involving lactose, further reducing the particle size, as shown in Table 4.

TABLE 4

| | |
|---|---|
| Formulation Composition (wt. %) | DLS Analysis |

| Nitazoxanide | F127 | Sucrose | D_{z} ± σ (nm) | PdI |
|---|---|---|---|---|
| 60 | 30 | 10 | 700 ± 30 | 0.390 |
| | 20 | 20 | 680 ± 20 | 0.388 |
| | 10 | 30 | 820 ± 50 | 0.353 |

The formulation which produced the smallest particle size (nitazoxanide/F127/sucrose 60/20/20 wt %) was taken forward for large scale formulation.

### Example 3: Large Scale Nitazoxanide Formulation

To produce the (nitazoxanide/F127/sucrose 60/20/20 wt %) on a large scale, the following procedure was followed.

1500 mg of F127 and 1500 mg of sucrose were dissolved in 135 mL of water to form the carrier material solution. Separately, 4.5 g of nitazoxanide was dissolved in 15 mL of anhydrous DMSO and heated to 60° C. The nitazoxanide-DMSO solution was then added to the excipient solution at a flow-rate of 5 mL/min using a peristaltic pump, whilst simultaneously homogenising the excipient solution at 9500 rpm using an IKA Yellowline D125 basic homogeniser. The subsequent dispersion was homogenised for a further two minutes following complete addition of the drug. The solution was immediately passed through the spray dryer at a flow-rate of 4 mL/minute (Buchi Mini B-290; Aspirator 100%, Nitrogen (cylinder pressure 3 bar) Q-flow gauge 45, Outlet temperature 60° C.). The powder was collected and dried in vacuo at ambient temperature to give a yield of 63%.

The procedure generated a fine, free-flowing powder that readily dispersed in saline. The dispersed suspension was analysed by DLS as outlined in the general method above, to find sub-micron particles with a D_{z} value of 698 nm (PdI of 0.205), highly consistent with the results of the small-scale example.

A sample of the powder was analysed by reverse phase high performance liquid chromatography (HPLC) using a photodiode array detector to quantify the drug content following the formulation process. An Agilent 1200 Series HPLC System equipped with a photodiode array detector was used for the analysis. The separation was achieved under isocratic conditions using an Agilent® Poroshell® 120 EC-C18, 2.7 μm, 4.6×50 mm column and a mobile phase consisting of 20 mM ammonium acetate aqueous buffer solution and acetonitrile (ammonium acetate_{(aq.)}/acetonitrile, 70/30). The column oven was set to 30° C. A flow rate of 0.5 mL/min and an injection volume of 2 μL was used. Detection was carried out using a wavelength of 425±4 nm. All calibration standards and formulation samples were prepared in H₂O/acetonitrile/DMSO (60/39/1), filtered through a 0.22 μm PTFE filter and immediately analysed following preparation. The nitazoxanide calibration curve was generated between 10 μg/mL and 50 μg/mL to give a linear correlation with a correlation coefficient of 0.9998.

The chromatogram consisted of two peaks: the main peak with a retention time of 4.2 min attributed to nitazoxanide and a minor peak with a retention time of 5.3 min attributed to the active metabolite, tizoxanide, formed by deacetylation of nitazoxanide during the formulation process. Analysis revealed a nitazoxanide composition of 57 wt. % within the formulation, comparing favourably to an expected value of 60 wt %.

A sample of the powder was analysed by ¹H-NMR to confirm the nitazoxanide content. Spectra were obtained using a Bruker® Avance® spectrometer operating at 400 MHz. Chemical shifts (δ) are reported in parts per million (ppm). The drug composition of the formulations was determined using a known concentration of benzyl methacrylate (BzMA) as an internal standard. Comparison of integrations between the resonances of the internal standard and known resonances of the drug enabled calculation of the moles of drug and therefore the mass of drug within the sample. Nitazoxanide formulations were run in DMSO-d₆ with a 10 mg/mL concentration of BzMA. The calculated drug composition was 56 wt % within the formulation, comparing favourably to an expected value of 60 wt %.

### Example 4: Niclosamide Formulations

Initially, niclosamide formulations targeted a drug composition of 50 wt. % and were obtained by screening with four excipients used for pulmonary administration of FDA-approved medicines: Pluronic F-127 (F127), hydroxypropyl methyl cellulose (HPMC), Tween 20 (polysorbate 20), and sucrose.

These formulations were obtained by flash nanoprecipitation, involving the rapid addition of a drug-Acetone/Ethanol (at 60° C.) solution to an aqueous solution of the carrier materials to generate suspensions. These suspensions were immediately spray dried to give powdered products with typical yields of 70%.

As a specific example, the niclosamide/HPMC/sucrose (50/25/25 wt %) formulation was produced as follows. 450 mg of HPMC and 450 mg of sucrose were dissolved in 30 mL of water to form the carrier material solution. Separately, 900 mg of niclosamide was dissolved in 15 mL of an 80:20 mixture of acetone:ethanol at 60° C. and rapidly added to the carrier material solution under stirring. The subsequent suspension was immediately passed through a spray dryer at a flow-rate of 5 mL/minute (Buchi Mini B-290; Aspirator 100%, Nitrogen (cylinder pressure 5 bar) Q-flow gauge 45, Outlet temperature 65° C.).

The particle size of each formulation was assessed by DLS as outlined in the general method above. The results are outline in Table 5 (below).

TABLE 5

| Niclosamide/Polymer/Sugar or | |
|---|---|
| Surfactant (50/25/25 wt %) Formulation | DLS Analysis |

| Polymer | Surfactant/sugar | D_{z} (nm) | PdI |
|---|---|---|---|
| F127 | HPMC | 4384 | 0.271 |
| HPMC | Sucrose | 728 | 0.458 |
| HPMC | Tween 20 | 2688 | 0.264 |

As a further specific example, the niclosamide/HPMC/sucrose (60/20/20 wt %) formulation was produced as follows. 120 mg of HPMC and 120 mg of sucrose were dissolved in 4 mL of water to form the carrier material solution. Separately, 360 mg of niclosamide was dissolved in 6 mL of an 80:20 mixture of acetone:ethanol at 60° C. and rapidly added to the carrier material solution under stirring. The subsequent suspension was immediately passed through a spray dryer at a flow-rate of 5 mL/minute (Buchi Mini B-290; Aspirator 100%, Nitrogen (cylinder pressure 5 bar) Q-flow gauge 45, Outlet temperature 65° C.).

TABLE 6

| | |
|---|---|
| Niclosamide/HPMC/Sucrose | |
| (60/20/20 wt %) Formulation | DLS Analysis |

| Polymer | Surfactant/sugar | D_{z} (nm) | PdI |
|---|---|---|---|
| HPMC | Sucrose | 697 | 0.433 |

As for the nitazoxanide formulations, the most promising formulation used a sugar, the disaccharide sucrose, rather than a surfactant. This formulation was taken forward for large-scale formulation experiments.

### Example 5: Large-Scale Niclosamide Formulation

The process of Example 4 was scaled up to produce the niclosamide/HPMC/sucrose (60/20/20 wt %) formulation on a larger scale and was modified to use a peristaltic pump to introduce the niclosamide solution and to include a sonication step to ensure effective dispersion of the niclosamide.

1.5 g of HPMC and 1.5 g of sucrose were dissolved in 100 mL of water to form the carrier material solution. Separately, 4.5 g of niclosamide was dissolved in 75 mL of an 80:20 mixture of acetone:ethanol at 60° C. and added to the carrier material solution via peristaltic pump at a rate of 10 mL/min under stirring. The tubing of the peristaltic pump was heated to prevent premature crystallization of the niclosamide. Once the addition was complete, the dispersion was sonicated in three 30 s bursts, with agitation in between each run to draw down any solid which had deposited onto the walls of the sample jar (Hieschler UP400s probe sonicator with H14 probe, cycle 1, amplitude 100%). The sonication effectively broke down any aggregates formed during mixing, preventing clogging of the spray-dryer. The solution was immediately passed through the spray dryer at a flow-rate of 4 mL/minute (Buchi Mini B-290; Aspirator 100%, Nitrogen (cylinder pressure 5 bar) Q-flow gauge 45, Outlet temperature 65° C.). The powder was collected and dried in vacuo at ambient temperature to give a yield of 78%.

A sample of the powder was analysed by ¹H-NMR to confirm the niclosamide content. The method corresponds with that used for the nitazoxanide formulation, with the exception that the niclosamide formulations were dissolved in DMF-d₇ with a 5 mg/mL concentration of BzMA.

### Example 6: Nebulisation of Formulations

0.9 wt % saline was used as the dispersant for nitazoxanide/F127/sucrose (60/20/20), niclosamide/HPMC/sucrose (50/25/25), and niclosamide/HPMC/sucrose (66/17/17) in amounts of 1 to 10 mg/mL. The dispersions were formed by mixing the required quantity of each solid composition with saline and shaking by hand for approximately three seconds.

A commercial vibrating membrane nebuliser was placed in a clamp on a retort stand, outlet facing downwards. A 50 mL skirted polypropylene centrifuge tube (pre-weighed with cap) was placed over the outlet to capture produced aerosol and the two pieces held together with Parafilm®. 10 mL of dispersion was pipetted onto the mesh of the nebuliser, which was switched on for 25 minutes. Further dispersion was added as required to prevent the mesh oscillating free of liquid at any point. After 25 minutes, the nebuliser was stopped and the centrifuge tube immediately and carefully capped to avoid loss of any aerosol before being weighed to determine the quantity of the dispersion that has passed through the nebuliser. The nebuliser was cleaned by passing 10 mL of 0.9% w/v saline solution and then triple rinsed with deionised water. The surfaces were gently dried with a low-lint tissue. The results are shown in Table 7.

TABLE 7

| | | Mass of | | Estimated |
|---|---|---|---|---|
| | Concen- | dispersion | Mass | time for 10 |
| | tration | passed | passed/time | g to pass |
| Formulation | (mg/mL) | (g) | (g/min) | (min) |
| nitazoxanide/F127/ | 1 | 7.670 | 0.31 | 32.59 |
| sucrose (60/20/20) | | | | |
| nitazoxanide/F127/ | 1 | 7.300 | 0.29 | 34.25 |
| sucrose (60/20/20) | | | | |
| nitazoxanide/F127/ | 1 | 5.809 | 0.23 | 43.03 |
| sucrose (60/20/20) | | | | |
| nitazoxanide/F127/ | 1 | 13.226 | 0.53 | 18.90 |
| sucrose (60/20/20) | | | | |
| nitazoxanide/F127/ | 1 | 7.270 | 0.29 | 34.39 |
| sucrose (60/20/20) | | | | |
| nitazoxanide/F127/ | 2 | 6.628 | 0.27 | 37.72 |
| sucrose (60/20/20) | | | | |
| nitazoxanide/F127/ | 2 | 6.306 | 0.25 | 39.64 |
| sucrose (60/20/20) | | | | |
| nitazoxanide/F127/ | 3 | 6.761 | 0.27 | 36.98 |
| sucrose (60/20/20) | | | | |
| nitazoxanide/F127/ | 5 | 5.898 | 0.24 | 42.39 |
| sucrose (60/20/20) | | | | |
| nitazoxanide/F127/ | 10 | 6.679 | 0.27 | 37.43 |
| sucrose (60/20/20) | | | | |
| nitazoxanide/F127/ | 10 | 7.391 | 0.30 | 33.82 |
| sucrose (60/20/20) | | | | |
| niclosamide/HPMC/ | 1 | 6.929 | 0.28 | 36.08 |
| sucrose (50/25/25) | | | | |
| niclosamide/HPMC/ | 1 | 6.784 | 0.27 | 36.85 |
| sucrose (50/25/25) | | | | |
| niclosamide/HPMC/ | 1 | 6.644 | 0.27 | 37.63 |
| sucrose (50/25/25) | | | | |
| niclosamide/HPMC/ | 1 | 7.264 | 0.29 | 34.42 |
| sucrose (50/25/25) | | | | |
| niclosamide/HPMC/ | 1 | 6.618 | 0.26 | 37.78 |
| sucrose (50/25/25) | | | | |
| niclosamide/HPMC/ | 1 | 7.597 | 0.30 | 32.91 |
| sucrose (50/25/25) | | | | |
| niclosamide/HPMC/ | 1 | 7.522 | 0.30 | 33.23 |
| sucrose (50/25/25) | | | | |
| niclosamide/HPMC/ | 1 | 6.766 | 0.27 | 36.95 |
| sucrose (50/25/25) | | | | |
| niclosamide/HPMC/ | 1 | 8.16 | 0.33 | 30.67 |
| sucrose (60/20/20) | | | | |
| niclosamide/HPMC/ | 1 | 8.25 | 0.33 | 30.30 |
| sucrose (60/20/20) | | | | |
| niclosamide/HPMC/ | 1 | 8.349 | 0.33 | 29.94 |
| sucrose (66/17/17) | | | | |

The particle sizes of the nebulised samples were analysed by DLS as described hereinbefore. In all cases, the small size and low polydispersity of the nanoparticles was either maintained or improved. The data for two formulations is shown in Table 8.

TABLE 8

| | nitazoxanide/F127/sucrose | | niclosamide/HPMC/sucrose | |
|---|---|---|---|---|
| | (60/20/20) | | (60/20/20) | |
| | Dz (nm) | PDI | Dz (nm) | PDI |
| Before | 918 | 0.439 | 966 | 0.400 |
| Nebulisation | | | | |
| After | 653 | 0.268 | 561 | 0.212 |
| Nebulisation | | | | |

### Example 7: Long Acting Injectable Formulations

An in vivo pharmacology study was completed in Sprague Dawley rats over 14 days, to determine the drug release kinetics of 50, 100, or 200 mg/kg doses of niclosamide administered as intramuscular injections of an aqueous dispersion of niclosamide nanoparticles.

The niclosamide/HPMC/sucrose (60/20/20 wt %) formulation was dispersed in a vehicle of 20 wt % HPMC, 20 wt % sucrose and 60 wt % water to produce aqueous dispersions at concentrations of 25, 50 or 100 mg/kg of niclosamide. Each animal received two 0.2 mL injections, one in each thigh, of one of the 25, 50, or 100 mg/kg of niclosamide dispersions for a total dose of 50, 100, or 200 mg/kg of niclosamide in each animal.

Blood samples were taken via tail vein bleed 1, 3, 6 and 24 hours post dose followed by bleeds at day 3, 4, 7 and 14. Niclosamide concentrations from the plasma samples were quantified utilising a previously validated liquid chromatography mass spectrometry (LCMS) method.

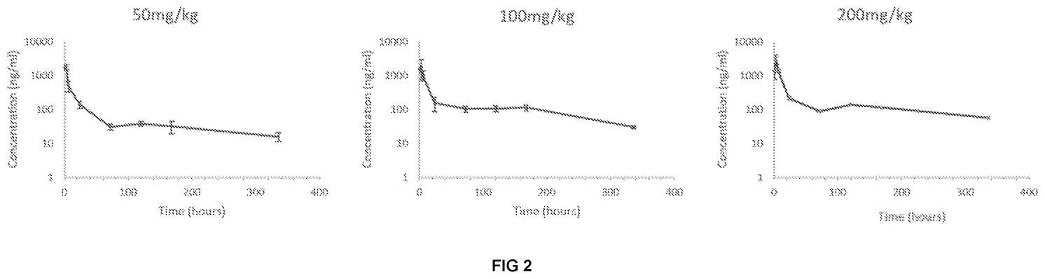

FIG. 2 shows a plot of the plasma concentration of niclosamide over time and demonstrates that the niclosamide release from the intramuscular depots to blood plasma was sustained over the 14-day period in all three dosage groups. The pharmacokinetic parameters of niclosamide for each study group are outlined in Table 9.

TABLE 9

| | Niclosamide Dose (mg/kg) | | |
|---|---|---|---|
| | 50 | 100 | 200 |
| Cₘₐₓ | 1408.64 | 2041.28 | 3125.34 |
| Tₘₐₓ | 2.50 | 2.00 | 3.00 |
| Cₘᵢₙ | 15.87 | 30.08 | 56.14 |
| C₂₄ | 140.21 | 158.39 | 215.01 |
| AUC_{0-14 days} | 24813.77 | 47532.39 | 58305.65 |

### Example 8: Further Niclosamide Formulations

Further niclosamide formulations using alternative excipients were created, the excipients being Plasdone C15, Kollidon 17PF, sucrose, and lactose.

These formulations were obtained by flash nanoprecipitation, involving the rapid addition of a drug-Acetone/Ethanol (at 60° C.) solution to an aqueous solution of the carrier materials to generate suspensions. These suspensions were immediately spray dried to give powdered products with typical yields of 70%.

As a specific example, the niclosamide/Plasdone C15/sucrose (50/25/25 wt %) formulation was produced as follows. 450 mg of HPMC and 450 mg of sucrose were dissolved in 30 mL of water to form the carrier material solution. Separately, 900 mg of niclosamide was dissolved in 15 mL of an 80:20 mixture of acetone:ethanol at 60° C. and rapidly added to the carrier material solution under stirring. The subsequent suspension was sonicated for a period of 60 seconds twice and then passed through a spray dryer at a flow-rate of 5 mL/minute (Buchi Mini B-290; Aspirator 100%, Nitrogen (cylinder pressure 5 bar) Q-flow gauge 45, Outlet temperature 65° C.).

The particle size of each formulation was assessed by DLS as outlined in the general method above. The results are outline in Table 10 (below).

TABLE 10

| Niclosamide/Polymer/Sugar | |
|---|---|
| (50/25/25 wt %) Formulation | DLS Analysis |

| Polymer | Sugar | D_{z} (nm) | PdI |
|---|---|---|---|
| Plasdone C15 | Sucrose | 373 | 0.307 |
| Plasdone C15 | Lactose | 395 | 0.256 |
| Kollidon 17PF | Sucrose | 562 | 0.281 |
| Kollidon 17PF | Lactose | 506 | 0.281 |

As a further specific example, the niclosamide/Plasdone C15/sucrose (60/20/20 wt %) formulation was produced as follows. 120 mg of Plasdone C15 and 120 mg of sucrose were dissolved in 4 mL of water to form the carrier material solution. Separately, 360 mg of niclosamide was dissolved in 6 mL of an 80:20 mixture of acetone:ethanol at 60° C. and rapidly added to the carrier material solution under stirring. The subsequent suspension was immediately passed through a spray dryer at a flow-rate of 5 mL/minute (Buchi Mini B-290; Aspirator 100%, Nitrogen (cylinder pressure 5 bar) Q-flow gauge 45, Outlet temperature 65° C.). This was also repeated using a reduced quantity of acetone:ethanol such that the niclosamide was at a concentration of 65 mg/mL, rather than a concentration of 60 mg/mL.

The particle size of each formulation was assessed by DLS as outlined in the general method above. The results are outline in Table 11 (below).

TABLE 11

| Niclosamide/HPMC/Sucrose | |
|---|---|
| (60/20/20 wt %) Formulation | DLS Analysis |

| Polymer | Sugar | D_{z} (nm) | PdI |
|---|---|---|---|
| Plasdone C15 | Sucrose | 395 | 0.256 |
| Plasdone C15 | Sucrose | 562 | 0.281 |

Further tests were performed on the niclosamide/Plasdone C15/sucrose (50/25/25 wt %) formulation, varying the conditions of the flash nanoprecipitation (i.e. the solvent system in which the niclosamide was dissolved, or increasing the aqueous volume into which the solvent system is mixed), the isolation of the nanoparticles (i.e. reducing the temperature of the spray dryer outlet) and/or of the dispersal of the resulting solid (i.e. dispersing by hand, rather than by vortex).

The results, shown below in Table 12, show that the process is robust with these excipients, producing high quality nanodispersions of niclosamide under a range of conditions.

TABLE 12

| Niclosamide/HPMC/Sucrose | |
|---|---|
| (50/25/25 wt %) Formulation | DLS Analysis |

| Condition Varied | D_{z} (nm) | PdI |
|---|---|---|
| Reduced spray dryer outlet temperature | 383 | 0.283 |
| Reduced spray dryer outlet temperature | 393 | 0.323 |
| and dispersal by hand | | |
| Increased aqueous volume during | 311 | 0.262 |
| nanoprecipitation | | |
| Butanone:ethanol (80:20) solvent system | 830 | 0.163 |

### Comparative Example 2: Wet Milling of Niclosamide

Niclosamide was wet milled with Plasdone C15 and sucrose in a mass ratio of 50:25:25 at a speed of 30 Hz. The resulting dispersion was subjected to DLS analysis, but the distribution of particle sizes was found to be multimodal, with significant quantities of material having sizes exceeding the maximum sizes accurately determinable by DLS.

### Example 9: Syringability of Niclosamide Formulations

A number of niclosamide formulations were tested for their maximum syringable concentration (i.e. the highest concentration, measured with respect to niclosamide, which a dispersion of the solid composition could attain while retaining stability and the ability to be passed through a 25 G needle). For each formulation tested, a 500 mg sample was measured into a vial before sufficient volume of the aqueous dispersant was added to produce the targeted niclosamide concentration. For example, a 500 mg sample of a niclosamide/Plasdone C15/sucrose (50/25/25 wt %) formulation, comprising 250 mg niclosamide, would be dispersed in 0.5 mL of aqueous dispersant when aiming to produce a dispersion with a niclosamide concentration of 500 mg/mL. The solid composition was fully dispersed using a vortex mixer for 30 to 180 seconds and the resulting aqueous dispersion was drawn into a 1 mL syringe. Any air was evacuated from the syringe before a 25 G needle was fitted. Finger pressure was applied to attempt to pass the dispersion through the needle. The samples were then left for 45 to 60 minutes before agitating using the vortex mixer and rechecking whether the sample could be passed through the 25 G needle to ensure that the dispersions were stable.

The results, summarised below in Table 13, show that the niclosamide formulations are syringable at extremely high concentrations of niclosamide.

TABLE 13

| | | | | Maximum |
|---|---|---|---|---|
| | Nanoprecipitation conditions | | | syringable |
| | | Volume | | NCL |
| | | ratio of | Total | concen- |
| | | solvent | solids | tration |
| | | system | wt/v | (mg · |
| Formulation | Solvent system | to water | % | mL⁻¹) |
| niclosamide/Plasdone | Acetone:ethanol | 1:2 | 4.00 | 300 |
| C15/sucrose (50/25/25 | (80:20) | | | |
| wt %) | | | | |
| niclosamide/Plasdone | Acetone:ethanol | 1:2 | 4.00 | 275 |
| C15/lactose (50/25/25 | (80:20) | | | |
| wt %) | | | | |
| niclosamide/Kollidon | Acetone:ethanol | 1:2 | 4.00 | 250 |
| 17PF/sucrose (50/25/ | (80:20) | | | |
| 25 wt %) | | | | |
| niclosamide/Kollidon | Acetone:ethanol | 1:2 | 4.00 | 225 |
| 17PF/lactose (50/25/ | (80:20) | | | |
| 25 wt %) | | | | |
| niclosamide/Plasdone | Acetone:ethanol | 1:3 | 3.75 | 275 |
| C15/sucrose (40/30/30 | (80:20) | | | |
| wt %) | | | | |
| niclosamide/Plasdone | Acetone:ethanol | 1:1.33 | 4.29 | 225 |
| C15/sucrose (60/20/20 | (80:20) | | | |
| wt %) | | | | |
| niclosamide/Plasdone | Acetone:ethanol | 1:1.44 | 4.43 | 225 |
| C15/sucrose (60/20/20 | (80:20) | | | |
| wt %) | | | | |
| niclosamide/Plasdone | Acetone:ethanol | 1:4 | 2.00 | 450 |
| C15/sucrose (50/25/25 | (80:20) | | | |
| wt %) | | | | |
| niclosamide/Plasdone | Acetone:ethanol | 1:2 | 4.00 | 375 |
| C15/sucrose (50/12.5/ | (80:20) | | | |
| 37.5 wt %) | | | | |
| niclosamide/Plasdone | Butanone:ethanol | 1:2 | 4.00 | 450 |
| C15/sucrose (50/25/25 | (80:20) | | | |
| wt %) | | | | |
| niclosamide/Plasdone | Butanone:ethanol | 1:4 | 2.00 | 575 |
| C15/sucrose (50/25/25 | (80:20) | | | |
| wt %) | | | | |

### Example 10: Large-Scale Niclosamide Formulation

The process of Example 8 was scaled up to produce the niclosamide/Plasdone 015/sucrose (50/25/25 wt %) formulation on a larger scale and was modified to use a peristaltic pump to introduce the niclosamide solution to the aqueous carrier solution.

1.75 g of Plasdone C15 and 1.75 g of sucrose were dissolved in 128.34 mL of water to form the carrier material solution. Separately, 3.5 g of niclosamide was dissolved in 46.67 mL of an 80:20 mixture of butanone:ethanol at 50° C. and added to the carrier material solution via peristaltic pump at a rate of 10 mL/min under stirring. The tubing of the peristaltic pump was heated to prevent premature crystallization of the niclosamide. Once the addition was complete, the dispersion was sonicated in three 30 s bursts, with agitation in between each run to draw down any solid which had deposited onto the walls of the sample jar (Hieschler UP400s probe sonicator with H14 probe, cycle 1, amplitude 100%). The sonication effectively broke down any aggregates formed during mixing, preventing clogging of the spray-dryer. The solution was immediately passed through the spray dryer at a flow-rate of 4 mL/minute (Buchi Mini B-290; Aspirator 100%, Nitrogen (cylinder pressure 5 bar) Q-flow gauge 45, Outlet temperature 65° C.). The resulting powder was found to have a D_{z} of 670 nm (PdI of 0.203) when dispersed at a concentration of 1 mg/mL in water, and was syringable up to niclosamide concentrations of 400 mg/mL.

### Example 11: Irradiation Stability Test

Injectable formulations are required to be sterilised prior to use and a common method for such sterilisation is gamma irradiation. Such irradiation can negatively affect the stability of materials. Therefore the long term stability of the formulations was tested following exposure to differing levels of radiation.

Samples of a niclosamide/HPMC/sucrose (60/20/20 wt %) formulation were exposed to 15, 25, or 35 kGy of gamma radiation and stored either at 25° C. and 60% relative humidity or at 40° C. and 75% relative humidity. Aliquots of each sample were taken at 0. 7, 28, and 56 days post sterilisation and analysed by DLS and HPLC. For each sample, aliquots were taken prior to irradiation and stored under identical conditions to act as a control. The DLS results are summarised in Tables 14 and 15, and show that, for each of the storage conditions, the unsterilized samples were stable over the entirety of the tested period and that there was no significant effect caused by exposure to radiation.

TABLE 14

| Stored at 25° C. and | | | Radiation Dose (kGy) | | | |
|---|---|---|---|---|---|---|
| 60% relative humidity | | | 0 | 15 | 25 | 35 |
| Time | 0 | Dz (nm) | 856 | 853 | 836 | 797 |
| (days) | | Pdl | 0.318 | 0.304 | 0.254 | 0.212 |
| | 7 | Dz (nm) | 871 | 893 | 853 | 1225 |
| | | Pdl | 0.253 | 0.332 | 0.202 | 0.423 |
| | 28 | Dz (nm) | 736 | 808 | 705 | 925 |
| | | Pdl | 0.206 | 0.215 | 0.206 | 0.222 |
| | 56 | Dz (nm) | 759 | 732 | 741 | 724 |
| | | Pdl | 0.253 | 0.232 | 0.23 | 0.185 |

TABLE 15

| Stored at 40° C. and | | | Radiation Dose (kGy) | | | |
|---|---|---|---|---|---|---|
| 75% relative humidity | | | 0 | 15 | 25 | 35 |
| Time | 0 | Dz (nm) | 856 | 853 | 836 | 797 |
| (days) | | Pdl | 0.318 | 0.304 | 0.254 | 0.212 |
| | 7 | Dz (nm) | 871 | 891 | 741 | 772 |
| | | Pdl | 0.253 | 0.313 | 0.244 | 0.243 |
| | 28 | Dz (nm) | 736 | 719 | 749 | 715 |
| | | Pdl | 0.206 | 0.024 | 0.214 | 0.189 |
| | 56 | Dz (nm) | 759 | 739 | 1067 | 709 |
| | | Pdl | 0.253 | 0.166 | 0.136 | 0.059 |

The HPLC data is summarised in Table 16, and found that sterilisation did not have a significant effect on the quantity of niclosamide present in each of the formulations.

TABLE 16

| | | | Niclosamide content | Average ± σ |
|---|---|---|---|---|
| Sample Details | | Run | (wt. %) | (wt. %) |
| Storage at 25° | 0 kGy | 1 | 55.0 | 54.7 ± 0.2 |
| C./60% RH | | 2 | 54.7 | |
| T = 56 days | | 3 | 54.5 | |
| | 15 kGy | 1 | 56.0 | 54.9 ± 1.0 |
| | | 2 | 55.1 | |
| | | 3 | 53.5 | |
| | 25 kGy | 1 | 54.9 | 54.6 ± 0.5 |
| | | 2 | 53.9 | |
| | | 3 | 55.1 | |
| | 35 kGy | 1 | 55.1 | 54.5 ± 0.5 |
| | | 2 | 54.5 | |
| | | 3 | 53.8 | |
| Storage at 40° | 0 kGy | 1 | 55.3 | 55.3 ± 0.4 |
| C./75% RH | | 2 | 54.8 | |
| T = 56 days | | 3 | 55.8 | |
| | 15 kGy | 1 | 55.7 | 54.4 ± 0.9 |
| | | 2 | 54.0 | |
| | | 3 | 53.6 | |
| | 25 kGy | 1 | 53.4 | 53.8 ± 1.1 |
| | | 2 | 52.7 | |
| | | 3 | 55.3 | |
| | 35 kGy | 1 | 54.5 | 54.2 ± 0.4 |
| | | 2 | 53.6 | |
| | | 3 | 54.5 | |

Overall, the formulations were found to be exceptionally stable under both of the tested storage conditions, regardless of the dosage of gamma irradiation to which they were exposed.

The foregoing examples have demonstrated the formation of solid compositions comprising nanoparticles of a pharmaceutically active compound exhibiting a Cₘₐₓ/EC₉₀ ratio of greater than 1 and/or is a member of the salicylanilide and thiazolide classes of drugs, optionally selected from tizoxanide prodrugs, such as nitazoxanide, and niclosamide, dispersed within a carrier material comprising at least one hydrophilic polymer and at least one sugar. In addition, the examples have proven methods for producing such solid compositions; and that such solid compositions may be dispersed in aqueous media to form aqueous dispersions of nanoparticles of a pharmaceutically active compound exhibiting a Cₘₐₓ/EC₉₀ ratio of greater than 1 and/or is a member of the salicylanilide and thiazolide classes of drugs. The suitability of said aqueous dispersions for administration by nebuliser has also been demonstrated, as has the long acting nature of injectable formulations comprising nanoparticles of a pharmaceutically active compound exhibiting a Cₘₐₓ/EC₉₀ ratio of greater than 1 and/or is a member of the salicylanilide and thiazolide classes of drugs, optionally selected from tizoxanide prodrugs, such as nitazoxanide, and niclosamide.

### Statements of the Disclosure:

Statement 1. A solid composition comprising a plurality of nanoparticles of a pharmaceutically active compound dispersed within a carrier material comprising at least one hydrophilic polymer and at least one sugar, wherein the pharmaceutically active compound is selected from compounds exhibiting a Cₘₐₓ/EC₉₀ ratio of greater than 1 and/or is a member of the salicylanilide and thiazolide classes of drugs.
Statement 2. The solid composition of Statement 1, wherein the pharmaceutically active compound is selected from compounds exhibiting a Cₘₐₓ/EC₉₀ ratio of greater than 2 and/or is a member of the salicylanilide and thiazolide classes of drugs, preferably the pharmaceutically active compound is selected from tizoxanide prodrugs, such as nitazoxanide, and niclosamide.
Statement 3. The solid composition of Statement 1 or Statement 2, wherein the at least one hydrophilic polymer is selected from polyvinyl alcohols, poloxamers, hydroxypropyl celluloses, and hydroxypropyl methyl celluloses.
Statement 4. The solid composition of any preceding Statement, wherein the at least one sugar is selected from monosaccharides, disaccharides, and oligosaccharides, preferably the at least one sugar is a disaccharide such as sucrose or lactose.
Statement 5. The solid composition of any preceding Statement, wherein:
   the pharmaceutically active compound is a thiazolide, preferably a tizoxanide prodrug, such as nitazoxanide;
   the at least one hydrophilic polymer is poloxamer; and
   the at least one sugar is selected from sucrose or lactose.
Statement 6. The solid composition of Statement 4 or Statement 5, wherein the solid composition comprises:
   50 to 60 wt % thiazolide, preferably a tizoxanide prodrug, such as nitazoxanide;
   10 to 30 wt % poloxamer; and
   10 to 30 wt % sucrose or lactose.
Statement 7. The solid composition of Statement 5, wherein the solid composition comprises:
   50 wt % thiazolide, preferably a tizoxanide prodrug, such as nitazoxanide;
   20 to 30 wt % poloxamer; and
   20 to 30 wt % lactose.
Statement 8. The solid composition of Statement 5, wherein the solid composition comprises:
   60 wt % thiazolide, preferably a tizoxanide prodrug, such as nitazoxanide;
   10 to 30 wt % poloxamer; and
   10 to 30 wt % sucrose.
Statement 9. The solid composition of any of Statements 1 to 4, wherein:
   the pharmaceutically active compound is a salicylanilide, preferably niclosamide;
   the at least one hydrophilic polymer is hydroxypropyl methyl cellulose; and
   the at least one sugar is sucrose.
Statement 10. The solid composition of Statement 9, wherein the solid composition comprises:
   50 to 70 wt % salicylanilide, preferably niclosamide;
   15 to 25 wt % hydroxypropyl methyl cellulose; and
   15 to 25 wt % sucrose.
Statement 11. A process for preparing a solid composition according to any of Statements 1 to 10, comprising the steps of:
   (a) providing an active solution comprising the pharmaceutically active compound in a water-miscible solvent;
   (b) providing a carrier material solution comprising one or more hydrophilic polymers and one or more sugars in an aqueous solvent;
   (c) mixing the solutions prepared in steps (a) and (b); and
   (d) removing the mixed solvent to produce the solid composition;
      wherein the pharmaceutically active compound is selected from compounds exhibiting a Cₘₐₓ/EC₉₀ ratio of greater than 1 and/or is a member of the salicylanilide and thiazolide classes of drugs.
Statement 12. The process of Statement 11, wherein the pharmaceutically active compound is selected from compounds exhibiting a Cₘₐₓ/EC₉₀ ratio of greater than 2 and/or is a member of the salicylanilide and thiazolide classes of drugs, preferably the pharmaceutically active compound is selected from tizoxanide prodrugs, such as nitazoxanide, and niclosamide.
Statement 13. The process of Statement 11 or Statement 12 wherein the at least one hydrophilic polymer is selected from polyvinyl alcohol, poloxamers, hydroxypropyl cellulose, and hydroxypropyl methyl cellulose.
Statement 14. The process of any of Statements 11 to 13, wherein the at least one sugar is selected from monosaccharides, disaccharides, and oligosaccharides, preferably the at least one sugar is a disaccharide, such as sucrose or lactose.
Statement 15. The process of any of Statements 11 to 14, wherein the water-miscible solvent is selected from dimethylsulfoxide, acetone, ethanol, or mixtures thereof.
Statement 16. The process of any of Statements 11 to 15, wherein the active solution is maintained at an elevated temperature prior to the mixing step.
Statement 17. The process of any of Statements 11 to 16, wherein the step of mixing the active solution and the carrier material solution additionally comprises homogenising and/or sonicating the dispersion.
Statement 18. The process of any of Statements 11 to 17, wherein the active solution and the carrier material solution are mixed in a ratio of about 1:9 to about 1:2.
Statement 19. The process of any of Statements 11 to 18, wherein the step of removing the mixed solvent comprises spray-drying.
Statement 20. The process of any of Statements 11 to 19, wherein:
   the pharmaceutically active compound is a thiazolide, preferably a tizoxanide prodrug, such as nitazoxanide;
   the at least one hydrophilic polymer is poloxamer;
   the at least one sugar is sucrose and/or lactose; and the water miscible solvent is dimethylsulfoxide.
Statement 21. The process of any of Statements 11 to 20, wherein:
   the pharmaceutically active compound is a salicylanilide, preferably niclosamide;
   the at least one hydrophilic polymer is hydroxypropyl methyl cellulose;
   the at least one sugar is sucrose; and
   the water miscible solvent is a mixture of ethanol and acetone.
Statement 22. A pharmaceutical composition comprising a solid composition according to any one of Statements 1 to 10, and optionally one or more pharmaceutically acceptable excipients.
Statement 23. The pharmaceutical composition of Statement 22, wherein the composition is a dry inhalable powder suitable for use with a dry powder inhaler.
Statement 24. The pharmaceutical composition of Statement 22, wherein the composition is a suspension of the solid composition, and optionally one or more pharmaceutically acceptable excipients, in a volatile propellant suitable for use with a pressurised metered-dose inhaler.
Statement 25. A solid composition according to any one of Statements 1 to 10, or a pharmaceutical composition according to any one of Statements 22 to 24, for use as a medicament.
Statement 26. A solid composition according to any one of Statements 1 to 10, or a pharmaceutical composition according to any one of Statements 22 to 24, for use in the treatment and/or prevention of viral infection, helminth infection, or protozoal infection, optionally wherein the viral infection is a coronavirus infection, such as SARS-CoV-2 infection.
Statement 27. A method of treating and/or preventing a viral infection, helminth infection, or protozoal infection, optionally wherein the viral infection is a coronavirus infection, such as SARS-CoV-2 infection, the method comprising administering a therapeutically effective amount of a solid composition according to any one of Statements 1 to 10, or a pharmaceutical composition according to any one of Statements 22 to 24, to a patient suffering from, or at risk of suffering from, the viral infection.
Statement 28. An aqueous dispersion comprising a plurality of nanoparticles of one or more pharmaceutically active compounds dispersed within an aqueous medium, wherein the pharmaceutically active compound is selected from compounds exhibiting a Cₘₐₓ/EC₉₀ ratio of greater than 1 and/or is a member of the salicylanilide and thiazolide classes of drugs, each nanoparticle being stabilised by the one or more hydrophilic polymers and/or the one or more sugars adsorbed to the surface of the nanoparticle.
Statement 29. The aqueous dispersion of Statement 28, wherein the pharmaceutically active compound is selected from compounds exhibiting a Cₘₐₓ/EC₉₀ ratio of greater than 2 and/or is a member of the salicylanilide and thiazolide classes of drugs, preferably the pharmaceutically active compound is selected from tizoxanide prodrugs, such as nitazoxanide, and niclosamide.
Statement 30. The aqueous dispersion of Statement 28 or Statement 29, wherein the aqueous phase comprises saline.
Statement 31. The aqueous dispersion of any of Statements 28 to 30, wherein the total solids content of the dispersion is in the range of 1 to 10 mg/mL.
Statement 32. A process for the preparation of an aqueous dispersion according to any of Statements 28 to 31, comprising dispersing a solid composition according to any one of Statements 1 to 10 in an aqueous medium.
Statement 33. A pharmaceutical composition comprising an aqueous dispersion according to any one of Statements 28 to 30, and optionally one or more pharmaceutically acceptable excipients.
Statement 34. An aqueous dispersion according to any one of Statements 28 to 30, or a pharmaceutical composition according to Statement 33, for use as a medicament.
Statement 35. An aqueous dispersion according to any one of Statements 28 to 30, or a pharmaceutical composition according to Statement 33, for use in the treatment and/or prevention of viral infection, helminth infection, or protozoal infection, optionally wherein the viral infection is a coronavirus infection, such as SARS-CoV-2 infection.
Statement 36. A method of treating and/or preventing a viral infection, helminth infection, or protozoal infection, optionally wherein the viral infection is a coronavirus infection, such as SARS-CoV-2 infection, the method comprising administering a therapeutically effective amount of an aqueous dispersion according to any one of Statements 28 to 30, or a pharmaceutical composition according to Statement 33, to a patient suffering from, or at risk of suffering from, the viral infection.
Statement 37. An intramuscularly-injectable pharmaceutically active compound formulation or a subcutaneously-injectable pharmaceutically active compound formulation comprising the solid composition of any of Statements 1 to 10, the aqueous dispersion of any of Statements 28 to 30, or the pharmaceutical composition of Statement 33.
Statement 38. The intramuscularly-injectable pharmaceutically active compound formulation of Statement 37, or the subcutaneously-injectable pharmaceutically active compound formulation of Statement 37, in depot form.
Statement 39. An intramuscularly-injectable pharmaceutically active compound formulation of Statement 37 or Statement 38, or a subcutaneously-injectable pharmaceutically active compound formulation of Statement 37 or Statement 38, for use as a medicament.
Statement 40. An intramuscularly-injectable pharmaceutically active compound formulation of Statement 37 or Statement 38, or a subcutaneously-injectable pharmaceutically active compound formulation of Statement 37 or Statement 38, for use in the treatment and/or prevention of viral infection, helminth infection, or protozoal infection, optionally wherein the viral infection is a coronavirus infection, such as SARS-CoV-2 infection.
Statement 41. A method of treating and/or preventing a viral infection, helminth infection, or protozoal infection, optionally wherein the viral infection is a coronavirus infection, such as SARS-CoV-2 infection, the method comprising administering a therapeutically effective amount of an intramuscularly-injectable pharmaceutically active compound formulation of Statement 37 or Statement 38, or a subcutaneously-injectable pharmaceutically active compound formulation of Statement 37 or Statement 38, to a patient suffering from, or at risk of suffering from, the viral infection.

1. A solid composition comprising a plurality of nanoparticles of a pharmaceutically active compound dispersed within a carrier material comprising at least one hydrophilic polymer and at least one sugar, wherein the pharmaceutically active compound is selected from nitazoxanide and niclosamide.

2. The solid composition of claim 1, wherein the at least one hydrophilic polymer is selected from polyvinyl alcohols, polyvinylpyrrolidones, poloxamers, hydroxypropyl celluloses, and hydroxypropyl methyl celluloses.

3. The solid composition of any preceding claim, wherein the at least one sugar is selected from monosaccharides, disaccharides, and oligosaccharides, preferably the at least one sugar is a disaccharide such as sucrose or lactose.

4. The solid composition of any preceding claim, wherein:
the pharmaceutically active compound is nitazoxanide;
the at least one hydrophilic polymer is poloxamer; and
the at least one sugar is selected from sucrose or lactose.

5. The solid composition of claim 3 or claim 4, wherein the solid composition comprises:
50 to 60 wt % nitazoxanide;
10 to 30 wt % poloxamer; and
10 to 30 wt % sucrose or lactose.

6. The solid composition of claim 5, wherein the solid composition comprises:
50 wt % nitazoxanide;
20 to 30 wt % poloxamer; and
20 to 30 wt % lactose.

7. The solid composition of claim 5, wherein the solid composition comprises:
60 wt % nitazoxanide;
10 to 30 wt % poloxamer; and
10 to 30 wt % sucrose.

8. The solid composition of any of claims 1 to 3, wherein:
the pharmaceutically active compound is niclosamide;
the at least one hydrophilic polymer is hydroxypropyl methyl cellulose; and
the at least one sugar is sucrose.

9. The solid composition of claim 8, wherein the solid composition comprises:
50 to 70 wt % niclosamide;
15 to 25 wt % hydroxypropyl methyl cellulose; and
15 to 25 wt % sucrose.

10. The solid composition of any of claims 1 to 3, wherein
the pharmaceutically active compound is niclosamide;
the at least one hydrophilic polymer is polyvinylpyrrolidone; and
the at least one sugar is sucrose or lactose.

11. The solid composition of claim 10, wherein the solid composition comprises:
40 to 70 wt % niclosamide;
10 to 30 wt % polyvinylpyrrolidone; and
15 to 40 wt % sucrose or lactose.

12. The solid composition of claim 11, wherein the solid composition comprises:
50 to 70 wt % niclosamide;
15 to 25 wt % polyvinylpyrrolidone; and
15 to 25 wt % sucrose or lactose.

13. A process for preparing a solid composition according to any of claims 1 to 12, comprising the steps of:
(a) providing an active solution comprising the pharmaceutically active compound in a water-miscible solvent;
(b) providing a carrier material solution comprising one or more hydrophilic polymers and one or more sugars in an aqueous solvent;
(c) mixing the solutions prepared in steps (a) and (b); and
(d) removing the mixed solvent to produce the solid composition;
wherein the pharmaceutically active compound is selected from nitazoxanide and niclosamide.

14. The process of claim 13 wherein the at least one hydrophilic polymer is selected from polyvinyl alcohol, polyvinylpyrrolidone, poloxamers, hydroxypropyl cellulose, and hydroxypropyl methyl cellulose.

15. The process of claim 13 or claim 14, wherein the at least one sugar is selected from monosaccharides, disaccharides, and oligosaccharides, preferably the at least one sugar is a disaccharide, such as sucrose or lactose.

16. The process of any of claims 13 to 15, wherein the water-miscible solvent is selected from dimethylsulfoxide, acetone, butanone, ethanol, or mixtures thereof.

17. The process of any of claims 13 to 16, wherein the active solution is maintained at an elevated temperature prior to the mixing step.

18. The process of any of claims 13 to 17, wherein the step of mixing the active solution and the carrier material solution additionally comprises homogenising and/or sonicating the dispersion.

19. The process of any of claims 13 to 18, wherein the active solution and the carrier material solution are mixed in a ratio of about 1:9 to about 1:2.

20. The process of any of claims 13 to 19, wherein the step of removing the mixed solvent comprises spray-drying.

21. The process of any of claims 13 to 20, wherein:
the pharmaceutically active compound is nitazoxanide;
the at least one hydrophilic polymer is poloxamer;
the at least one sugar is sucrose and/or lactose; and
the water miscible solvent is dimethylsulfoxide.

22. The process of any of claims 13 to 20, wherein:
the pharmaceutically active compound is niclosamide;
the at least one hydrophilic polymer is hydroxypropyl methyl cellulose or polyvinylpyrrolidone;
the at least one sugar is sucrose or lactose; and
the water miscible solvent is a mixture of ethanol and acetone or is a mixture of ethanol and butanone.

23. A pharmaceutical composition comprising a solid composition according to any one of claims 1 to 12, and optionally one or more pharmaceutically acceptable excipients.

24. The pharmaceutical composition of claim 23, wherein the composition is a dry inhalable powder suitable for use with a dry powder inhaler.

25. The pharmaceutical composition of claim 23, wherein the composition is a suspension of the solid composition, and optionally one or more pharmaceutically acceptable excipients, in a volatile propellant suitable for use with a pressurised metered-dose inhaler.

26. A solid composition according to any one of claims 1 to 12, or a pharmaceutical composition according to any one of claims 23 to 25, for use as a medicament.

27. A solid composition according to any one of claims 1 to 12, or a pharmaceutical composition according to any one of claims 23 to 25, for use in the treatment and/or prevention of viral infection, helminth infection, or protozoal infection, optionally wherein the viral infection is a coronavirus infection, such as SARS-CoV-2 infection.

28. A method of treating and/or preventing a viral infection, helminth infection, or protozoal infection, optionally wherein the viral infection is a coronavirus infection, such as SARS-CoV-2 infection, the method comprising administering a therapeutically effective amount of a solid composition according to any one of claims 1 to 12, or a pharmaceutical composition according to any one of claims 23 to 25, to a patient suffering from, or at risk of suffering from, the viral infection.

29. An aqueous dispersion comprising a plurality of nanoparticles of one or more pharmaceutically active compounds dispersed within an aqueous medium, wherein the pharmaceutically active compound is selected from nitazoxanide and niclosamide, each nanoparticle being stabilised by the one or more hydrophilic polymers and/or the one or more sugars adsorbed to the surface of the nanoparticle.

30. The aqueous dispersion of claim 29, wherein the aqueous phase comprises water, saline, or phosphate buffered saline.

31. The aqueous dispersion of claim 29 or claim 30, wherein the concentration of the pharmaceutically active compound in the dispersion is in the range of 1 to 800 mg/mL, 10 to 600 mg/mL, 225 to 575 mg/mL, or 300 to 500 mg/mL.

32. A process for the preparation of an aqueous dispersion according to any of claims 29 to 31, comprising dispersing a solid composition according to any one of claims 1 to 12 in an aqueous medium.

33. A pharmaceutical composition comprising an aqueous dispersion according to any one of claims 29 to 31, and optionally one or more pharmaceutically acceptable excipients.

34. An aqueous dispersion according to any one of claims 29 to 31, or a pharmaceutical composition according to claim 33, for use as a medicament.

35. An aqueous dispersion according to any one of claims 29 to 31, or a pharmaceutical composition according to claim 33, for use in the treatment and/or prevention of viral infection, helminth infection, or protozoal infection, optionally wherein the viral infection is a coronavirus infection, such as SARS-CoV-2 infection.

36. A method of treating and/or preventing a viral infection, helminth infection, or protozoal infection, optionally wherein the viral infection is a coronavirus infection, such as SARS-CoV-2 infection, the method comprising administering a therapeutically effective amount of an aqueous dispersion according to any one of claims 29 to 31, or a pharmaceutical composition according to claim 33, to a patient suffering from, or at risk of suffering from, the viral infection.

37. An intramuscularly-injectable pharmaceutically active compound formulation or a subcutaneously-injectable pharmaceutically active compound formulation comprising the solid composition of any of claims 1 to 12, the aqueous dispersion of any of claims 29 to 31, or the pharmaceutical composition of claim 33.

38. The intramuscularly-injectable pharmaceutically active compound formulation of claim 37, or the subcutaneously-injectable pharmaceutically active compound formulation of claim 37, in depot form.

39. An intramuscularly-injectable pharmaceutically active compound formulation of claim 37 or claim 38, or a subcutaneously-injectable pharmaceutically active compound formulation of claim 37 or claim 38, for use as a medicament.

40. An intramuscularly-injectable pharmaceutically active compound formulation of claim 37 or claim 38, or a subcutaneously-injectable pharmaceutically active compound formulation of claim 37 or claim 38, for use in the treatment and/or prevention of viral infection, helminth infection, or protozoal infection, optionally wherein the viral infection is a coronavirus infection, such as SARS-CoV-2 infection.

41. A method of treating and/or preventing a viral infection, helminth infection, or protozoal infection, optionally wherein the viral infection is a coronavirus infection, such as SARS-CoV-2 infection, the method comprising administering a therapeutically effective amount of an intramuscularly-injectable pharmaceutically active compound formulation of claim 37 or claim 38, or a subcutaneously-injectable pharmaceutically active compound formulation of claim 37 or claim 38, to a patient suffering from, or at risk of suffering from, the viral infection.
